(19) United States
(12) Patent Application Publication
(10) Pub. No.: US 2024/0423474 A1
(43) Pub. Date: Dec. 26, 2024

(54) SYSTEM AND METHOD FOR INTRAOPERATIVE LIFETIME IMAGING

(71) Applicant: The General Hospital Corporation, Boston, MA (US)

(72) Inventors: Anand T.N. Kumar, Arlington, MA (US); Rahul Pal, Allston, MA (US); Murali Krishnamoorthy, Somerville, MA (US)

(73) Assignee: The General Hospital Corporation, Boston, MA (US)

(21) Appl. No.: 18/705,368

(22) PCT Filed: Oct. 25, 2022
(86) PCT No.: PCT/US2022/078656

§ 371 Date: Apr. 26, 2024

Related U.S. Application Data
(60) Provisional application No. 63/272,847, filed on Oct. 28, 2021.
(60) Provisional application No. 63/366,483, filed on Jun. 16, 2022.

(51) Int. Cl. A61B 5/00; G16H 20/40; G16H 30/40; G16H 50/20

(52) U.S. Cl. CPC A61B 5/0071; G16H 20/40; G16H 30/40; G16H 50/20; A61B 2505/05

(57) ABSTRACT

A method and system for assessing tissue to determine a presence or absence of cancer cells. The method includes acquiring fluorescence lifetime (FLT) data from tissue and processing the FLT data to determine a FLT signal at each of a plurality of locations across the tissue. The method also includes determining FLT data at any of the plurality of locations above a threshold indicative a presence of cancer cells and generating a report indicating any of the plurality of locations above the threshold as indicative the presence of cancer cells.

### CROSS-REFERENCE TO RELATED APPLICATIONS

This application is based on, claims priority to, and incorporates herein by reference in their entirety U.S. Provisional Application Ser. No. 63/272,847, filed Oct. 28, 2021, and 63/366,483, filed Jun. 16, 2022.

### STATEMENT REGARDING FEDERALLY SPONSORED RESEARCH

N/A

### BACKGROUND

The present disclosure relates generally to systems and methods for assessing tissue intraoperatively. More particularly, the present disclosure provides systems and methods for intraoperative examination of tissue and identification of target cells in-vivo to further guide the operative procedure.

Despite major advancements in surgical oncology, the positive margin rate for cancer resections remains high. The existence of tumor positive margins in surgical resections leads to poor clinical outcomes. Surgeons rely on palpation, visual inspection, or standardized measures to determine tumor/normal boundary during surgery. These methods do not guarantee complete resections, leading to local recurrence and poor overall survival. Post-resection margin assessment is performed by inspection of gross specimens, plain radiographs, or histopathology, which capture margin status in only a fraction of the specimen, potentially missing viable cancer. There is a need for imaging techniques that are sensitive enough to detect any cancer left behind in the surgical bed, and to assess margin status in entire specimen post-surgery, thereby ensuring complete tumor removal, reducing cancer recurrence rates, and improving overall survival and the quality of life of patients.

With this in mind, a variety of efforts have been made to use imaging to assess the resection site in vivo. With this in mind, fluorescence imaging methods have shown promise for margin guidance using fluorescent probes and are attractive due to the low cost, high sensitivity, relatively simple instrumentation and use of non-ionizing radiation. Thus, many fluorescent contrast agents and imaging systems have been developed in past couple of decades for cancer diagnosis, image guided surgery and drug development.

Most of these imaging applications utilize a dye and the knowledge that cancer cells have a higher uptake rate than healthy cells. However, background fluorescence from normal tissue and non-specific probe uptake reduces tumor contrast, resulting in poor sensitivity and specificity. Traditional imaging systems detect total fluorescence intensity, which cannot easily distinguish fluorescence signals arising from tumor-bound probe from non-specific fluorescence. Furthermore, because fluorescence intensity depends on probe uptake, and hence on tumor size, smaller tumors are harder to detect against non-specific background.

Thus, some of these agents are tumor targeting. For example, the use of therapeutic antibodies for receptors overexpressed in cancers is a powerful approach for tumor targeting, given that antibodies are more likely to be retained in cancer cells and are less complex and less expensive to manufacture. One such agent that has shown great promise is pantitumumab-IRDye800CW, a conjugate of the FDA approved therapeutic antibody for the epidermal growth factor receptor (EGFR), panitumumab, with IRdye800CW, an NIR dye that has been tested in multiple human trials. EGFR is a prospective target for fluorescence imaging because it is overexpressed in several cancers, including head and neck, lung, gliomas, and metastatic colorectal cancer (mCRC). Several recent clinical trials have shown that panitumumab-IRDye800CW is safe for human use and can enhance tumor contrast during fluorescence guided-surgical resections and differentiate benign from metastatic lymph nodes in patients with head and neck squamous cell carcinoma (HNSCC).

Thus, despite many advances in identifying new cancer-specific molecular targets and imaging probes, no agent has been widely adopted for clinic use, primarily due to poor pharmacokinetics and relatively low tumor uptake. Conventional fluorescence imaging systems detect total fluorescence intensity re-emitted from the sample. Fluorescence intensity depends on a product of probe concentration and fluorescence lifetime, and therefore cannot easily distinguish tumor specific fluorescence from non-specific dye accumulation of probe in healthy tissue. Further, fluorescence intensity is affected by tumor size and probe uptake, making it difficult to detect small lesions in the surgical bed with sufficient signal to background ratio. Fluorescence intensity is also strongly affected by tissue attenuation and system-specific measurement parameters, including the power of the illuminating light, detector or camera sensitivity and response characteristics, and spurious leakage of ambient light. As a result, fluorescence intensity measurements cannot be readily compared across multiple specimens, subjects, and imaging systems on an absolute scale, thereby hindering standardization and ease of adoption.

Despite over 30 years of effort in developing new imaging agents and many promising clinical trials, cancer cell-specific labelling has not yet been demonstrated using exogenous agents in humans. Non-specific probe accumulation in normal or benign tissue remains a major problem that significantly lowers relative tumor brightness compared to background and results in poor signal to noise ratio, low specificity (false positives) and low sensitivity (false negatives).

In attempts to overcome issues such as these, some have paired specially-designed hardware with a particular imaging agent/dye (using targeted or untargeted tracers). Also, specialized imaging techniques have been explored to overcome the poor targeting specificity of molecular imaging agents, including administration of a concurrent loading dose to improve probe uptake, paired-agent imaging with targeted and untargeted probes, or referencing schemes. However, these techniques have either not been demonstrated to be sufficiently robust for widespread clinical adoption, or have an arduous path to clinical translation due to the need for administering multiple agents. Thus, clinical implementation of these strategies face significant financial and regulatory barriers, and has yet to establish superior and consistent results.

Therefore, it would be desirable to have a system and method for in-vivo analysis of tissue that is capable of analyzing a substantially large volume of tissue with sufficient accuracy to provide clinical certainty of tissue pathology.

### SUMMARY

The present disclosure overcomes the aforementioned drawbacks by providing systems and methods for intraoperative tissue assessment that does not require new dyes or specialized tracers, and does not require pairings of specialized hardware with specialized dyes or tracers. The present disclosure provides systems and methods for assessing intraoperative tissue, such as resection beds and margins, using fluorescence lifetime (FLT) imaging. That is, the present disclosure recognizes that FLT is longer in cancer cells compared to non-specific dye in normal tissue. Thus, systems and methods are provided whereby contrast between cancerous and normal tissue are dramatically distinguished, with greater sensitivity and specificity than traditional dye and imaging systems that rely on tumor uptake. The systems and methods provided herein can assess FLT in absolute units (nanoseconds) that are not system-dependent and are unaffected by light-tissue interactions such as scattering an absorption. Thus, systems and methods are provided that facilitate robust standardization in intraoperative, in vivo, tissue assessment.

In accordance with one aspect of the present disclosure, a method is provided for assessing tissue to determine a presence or absence of cancer cells. The method includes acquiring fluorescence lifetime (FLT) data from tissue and processing the FLT data to determine a FLT signal at each of a plurality of locations across the tissue. The method also includes determining FLT data at any of the plurality of locations above a threshold indicative a presence of cancer cells and generating a report indicating any of the plurality of locations above the threshold as indicative the presence of cancer cells.

In accordance with another aspect of the present disclosure, a medical imaging system is provided that includes an optical source configured to deliver light to tissue, a detector configured to receive light fluoresced by the tissue and produce fluorescence lifetime (FLT) data. A processor is configured to analyze the FLT data to determine a presence or absence of cancer in the tissue and generate a report indicating a spatial location of any cancer determined as present in the tissue. The system also includes a display configured to display the report to guide a surgical procedure to remove the cancer.

The foregoing and other advantages of the inventions will appear in the detailed description that follows. In the description, reference is made to the accompanying drawings.

### BRIEF DESCRIPTION OF THE DRAWINGS

Implementations of the invention will become more apparent from the detailed description set forth below when taken in conjunction with the drawings, in which like elements bear like reference numerals.

FIG. 1 is diagram of one, non-limiting example of a system in accordance with the present disclosure.

FIG. 2 is a flow chart setting forth some, non-limiting example steps of a process in accordance with the present disclosure that may utilize a system such as described with respect to FIG. 1.

FIG. 3A is a graph illustrating derivation of non-linear fits in accordance with the present disclosure.

FIG. 3B is a graph showing the quasi-time domain (QTD) for the same two lifetimes.

FIG. 3C is a graph of SNR for a range of lifetimes and noise levels using standard TD data.

FIG. 3D is a graph of SNR for varying lifetimes and noise levels using QTD data and showing significant improvement in SNR compared to standard TD data for the entire range of lifetimes and noise levels simulated.

FIG. 4A is a fluorescence lifetime microscopy (FLIM) image of a thin tissue slice from a patient with skin cancer, injected with ICG 24 hours prior to surgery.

FIG. 4B is a standard histological (hematoxylin and eosin) stained image of the sample of FIG. 4A.

FIG. 4C is a boxplot showing the lifetimes of various tissue types within the tissue section of FIG. 4B and a cutoff lifetime level (dashed line) above which the tissue is cancerous.

FIG. 5A is a boxplot showing the fluorescence lifetimes of liver hepatocellular carcinoma (HCC) and normal tissue, resected from patients injected with ICG and a cutoff lifetime level (dashed line) above which the tissue is cancerous.

FIG. 5B is a boxplot showing the fluorescence lifetimes of oral SCC and normal tissue, resected from patients injected with ICG and a cutoff lifetime level (dashed line) above which the tissue is cancerous.

FIG. 6A is a color photograph of resected tissue from a patient with cutaneous SCC including cancerous and normal tissue.

FIG. 6B is an image showing the resected tissue of FIG. 6A with a lifetime data overlay.

FIG. 6C is an image showing the resected tissue of FIG. 6A with a mask.

FIG. 6D is an image showing the resected tissue of FIG. 6A with a fluorescence intensity data overlay.

FIG. 7A is an image of a head and neck specimen resected from a patient with oral SCC.

FIG. 7B is a microscopic FLT image from small sections within the positive and negative LNs, showing the significantly longer lifetime in the positive LN.

FIG. 7C is a wide-field fluorescence intensity image of the specimen of FIGS. 7A and 7B

FIG. 7D is a wide-field fluorescence lifetime image indicating that the FLT values within the positive LN of FIG. 7A are significantly and uniformly longer than the FLTs of the negative LN, which stands in stark contrast to what the intensity image of FIG. 7C shows.

FIG. 8A is a graph of time domain (TD) fluorescence signals measured from liver HCC specimens freshly resected from patients systemically injected with ICG 24 to 48 hours prior to surgery.

FIG. 8B is a graph of time domain (TD) fluorescence signals measured from oral SCC cancer surgical specimens freshly resected from patients systemically injected with ICG 24 to 48 hours prior to surgery.

FIG. 8C is histogram showing fluorescence intensity data in tumor (red) and normal (green) tissue.

FIG. 8D is histogram showing FLT data in tumor (red) and normal (green) tissue.

FIG. 9 is a box-and-whisker plot showing the FLT distribution of multiple tumor types (HCC, mCRC and OSCC) compared with the FLTs of ICG in various normal tissue types.

FIG. 10A is graph showing representative TD fluorescence decay curves of panitumumab-IRDye800CW (gray solid), IgG-IRDye800CW (black dashed) and PBS (gray dashed) in cancer cells.

FIG. 10B is graph showing representative TD fluorescence decay curves of panitumumab-IRDye800CW (gray solid) and IgG-IRDye800CW (black dashed) in culture media, and the stock solution of panitumumab-IRDye800CW in PBS (gray dotted).

FIG. 10C is a set of confocal microscopy images of fluorescence intensity and FLT of cancer cells after incubation with panitumumab-IRDye800CW (100 g), IgG-IRDye800CW (100 μg) or PBS at 370 C for 2 hours.

FIG. 10D is set of widefield FLT maps of culture media collected after incubation of imaging probes with cancer cells and panitumumab-IRDye800CW in PBS.

FIG. 11A is set of high resolution images, including FLIM, IHC, and H&E stained images, showing enhanced FLT in tumor areas with high EGFR expression.

FIG. 11B is set of images, including FLIM, IHC, and H&E stained images, showing an expanded view within the noted area of FIG. 11A.

FIG. 11C is set of images, including FLIM, IHC, and H&E stained images, showing an expanded view within the noted area of FIG. 11B.

FIG. 12A is a set of representative confocal fluorescence intensity and FLIM images, along with corresponding H&E and EGFR IHC images, from clinical specimens with low magnification.

FIG. 12B is the set of images of FIG. 12A at higher magnification.

FIG. 12C is another set of images of FIG. 12A at a higher magnification.

FIG. 13A show EGFR expression (% area positive for EGFR) IHC images in ROIs from muscle, salivary gland and tumor, respectively, shown in an increasing order of expression.

FIG. 13B is a set of confocal fluorescence intensity images and intensity histograms corresponding to the images of FIG. 13A.

FIG. 13C is a set of confocal FLIM images and FLT histograms of the same ROIs as in FIG. 13A showing an increasing trend of FLT values with increasing EGFR expression.

FIG. 13D is a scatter plot of average fluorescence intensity versus the percent area positive for EGFR in IHC across all ROIs imaged.

FIG. 13E is a graph showing average fluorescence intensities in EGFR negative and positive pixels obtained from co-registered IHC and FLIM images.

FIG. 13F is a scatter plot of average FLT versus percent area positive for EGFR across all ROIs.

FIG. 13G is a graph of average FLTs in EGFR negative and positive pixels of the same ROIs. The bar graphs are plotted as mean with standard deviation.

FIG. 14A is a photograph of a tissue sample.

FIG. 14B is an image of an H&E stain of the tissue sample of FIG. 14A.

FIG. 14C is an intensity image of the tissue sample of FIG. 14A

FIG. 14D is an image showing Panitumumab-IRDye800CW amplitude in the tissue sample of FIG. 14A.

FIG. 14E is an image showing tissue autofluorescence amplitude in the tissue sample of FIG. 14A.

FIG. 14F is a widefield FLT image of the specimen showing the tumor boundary (dotted line) from the co-registered H&E image presented in FIG. 14B.

FIG. 14G is a FLIM image of a rectangular region shown in FIGS. 14C-14F.

FIG. 14H is a graph of distribution of fluorescence intensity data from the sample.

FIG. 14I is a graph of distribution of spectral unmixing data from the sample.

FIG. 14J is a graph of distribution of fluorescence lifetime data from the sample.

FIG. 14K is set of ROC curves for tumor versus normal tissue classification using FLT (black solid), fluorescence intensity (gray dashed), and spectral unmixing (gray solid) based on the H&E ground truth.

FIG. 15A is a graph of distributions of fluorescence intensity for sarcoma tumors and normal tissue.

FIG. 15B is a graph of distributions of FLT for sarcoma tumors and various normal tissue types.

FIG. 15C is a graph of mean fluorescence intensity for the sarcoma tumors and various normal tissue types across 7 different patients.

FIG. 15D is a graph of mean FLT for the sarcoma tumors and normal tissue across seven different patients.

FIG. 16A is a graph showing a distribution of fluorescence intensity in oral cancers and normal oral tissue from 8 patients.

FIG. 16B is a graph showing a distribution of FLT data in oral cancers and normal oral tissue from 8 patients.

FIG. 16C is a graph of mean tumor and normal intensity across six HN cancer patients.

FIG. 16D is a graph of mean tumor and normal FLT data across six HN cancer patients.

FIG. 17A is a plot of sensitivity versus false positive rate for FLT and intensity based tumor vs. normal classification across 10 sarcoma patients.

FIG. 17B is a plot of sensitivity versus false positive rate for FLT and intensity based tumor vs. normal classification across 8 head and neck cancer patients.

FIG. 18A is an example fluorescence intensity image of bacteria expressing five iRFP variants (iRFP670, 682, 702, 713 and 720).

FIG. 18B is an example fluorescence lifetime image of bacteria expressing five iRFP variants (iRFP670, 682, 702, 713 and 720).

FIG. 18C is a graph showing normalized excitation spectra of three iRFP variants.

FIG. 18D is a graph showing emission spectra of iRFP670, 702 and 720.

FIG. 18E is a graph of time domain (TD) fluorescence signal of iRFP670, 702 and 720 in bacteria.

FIG. 18F is histogram of lifetimes derived from FIG. 18B.

FIG. 18G is an example fluorescence intensity image of three MTLn3 tumors expressing iRFP670, 702 and 720 located in the mammary fat pad of a female nude mouse.

FIG. 18H is an example fluorescence lifetime image of the three MTLn3 tumors expressing iRFP670, 702 and 720 located in the mammary fat pad of the female nude mouse.

FIG. 19A is a fluorescence intensity reflectance image from a mouse injected with bone and kidney targeting fluorescent dyes.

FIG. 19B shows decay amplitudes obtained from a bi-exponential fit of the time domain data with fixed lifetimes of 0.5 ns (green) and the 0.65 ns (red)

FIG. 19C shows 3D reconstruction using the using the asymptotic time domain (ATD) approach clearly separates skeleton and localizes the kidneys.

FIG. 19D shows amplitude maps in situ (no skin).

### DETAILED DESCRIPTION

The present disclosure recognizes that the fluorescence lifetimes (FLTs) of fluorescent dyes are significantly longer in tumor cells than the FLTs of the same dyes in healthy tissue. With this in mind, the present disclosure provides systems and methods for intraoperative tissue analysis that can distinguish tumors from healthy tissue with a consistency and specificity not realized in prior attempts to do so. Thus, the present disclosure recognizes that FLT, which can be measured in absolute units (typically nanoseconds), is a parameter that is robust to measurement conditions and can be used to alleviate many of the shortcomings of prior efforts at creating a robust, intraoperative tissue assessment tool.

The systems and methods provided herein are flexible. The systems and methods provided herein do not require a specific paring of hardware with a particular dye or targeting agent. In one, non-limiting example, a fluorescently tagged EGFR-antibody can be used, but other dyes or fluorescent tagging mechanisms can be utilized. In accordance with one non-limiting aspect of the disclosure, the FLT imaging can utilize the near infrared (NIR) spectrum, but other wavelengths may also be used. The systems and methods provided herein can provide a dramatic specificity and sensitivity improvement over standard fluorescence intensity-based methods for distinguishing tumors form normal tissue in situ and in vivo.

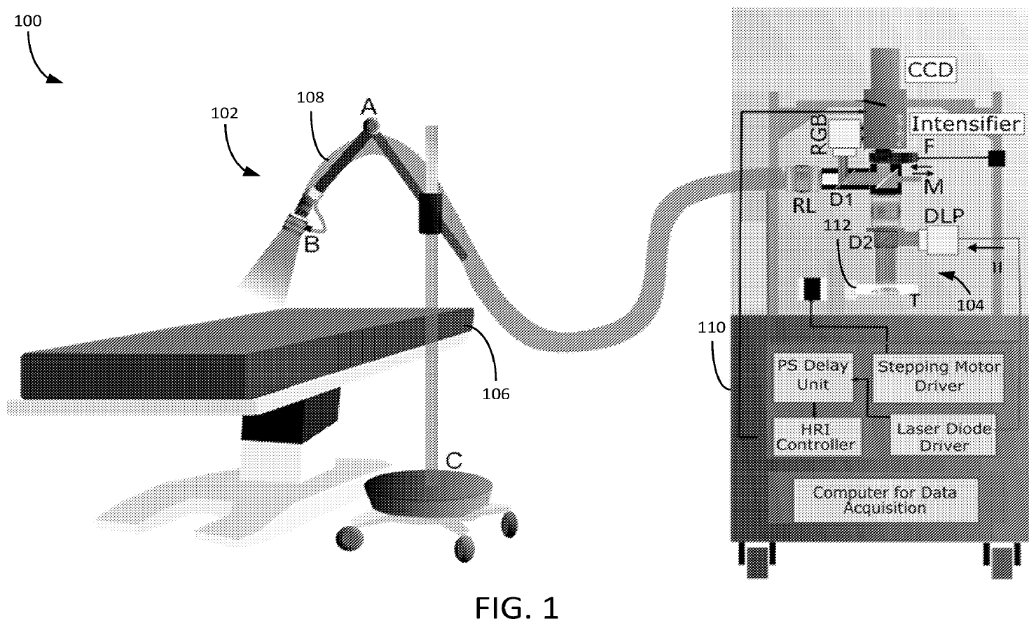

Referring to FIG. 1, one, non-limiting example of a system 100 that may be used in accordance with the present disclosure is illustrated. In the illustrated, non-limiting example, the system 100 may be a time domain (TD) imaging platform configured for both in vivo imaging and ex vivo or in vitro imaging. in particular, the illustrated, non-limiting system 100 includes an in vivo imaging sub-system 102 and an in vitro or ex vivo imaging sub-system 104.

In the illustrated configuration, the in vivo imaging sub-system 102 is designed to direct light from a surgical bed 106 to an optional fiber bundle 108, which is then collected by a relay lens (RL) and split via a dichroic mirror (D1) into one or more cameras (RGB). In one non-limiting example, images may be collected directly by a camera and the camera may be designed for wavelengths less than a threshold, for example, 650 nm. In this case, an intensified camera (CCD/Intensifier) may be included that are designed for wavelengths over the threshold, for example, 650 nm. Also, a second camera may be included that is not time gated, and collects intensity images in parallel, real-time. Alternatively, intensity data may be acquired using only a single camera by summing the time gated data, cumulatively.

A mirror housing (M) may be attached to the Intensifier/CCD and may be configured to be remotely switched to receive light from the fiber bundle 108 or from the specimen stage. Fluorescence or NIR excitation can be collected using, for example, a filter wheel (F) attached to the ICCD. A fiber delivers light (for example, 780 nm light) into both a digital light projector (DLP) via a dichroic (D2) (for example, 800 nm) for specimen illumination, and to the surgical bed 106 via a port in an objective lens (B). These wavelengths are simply examples, and other wavelengths can be utilized. In particular, as will be described, the near infrared (NIR) spectrum may be utilized. In this non-limiting example of NIR light, the light can penetrate up to 5-10 cm into the tissue, which can be advantageous for assessing even tumor that is beneath several cm thick tissue layer.

As will be described, the system 100 can be configured for a wide field-of-view (FOV) while providing micron resolution. The fiber bundle is mounted on a flexible articulating arm (A) attached to a portable stand (C). The arm A can be positioned for a desired view of the surgical bed 106. In this way, an in vivo probe is provided that can be hand-directed or hand-held for manipulation about the surgical site.

As illustrated, the system 100 can be integrated into a cart or rack 110, which can include the in vitro or ex vivo imaging sub-system 104. The in vitro or ex vivo imaging sub-system 104 can be controlled by a stepping motor driver that can control positioning of a sample chamber 112. The system 100 may also include, as illustrated a laser diode driver, a PS delay unit, and an HRI controller configured to coordinate delivery of the laser illumination. The system 100 may also include a computer system or processor that is configured for data acquisition, data processing, and report generation in accordance with the present disclosure.

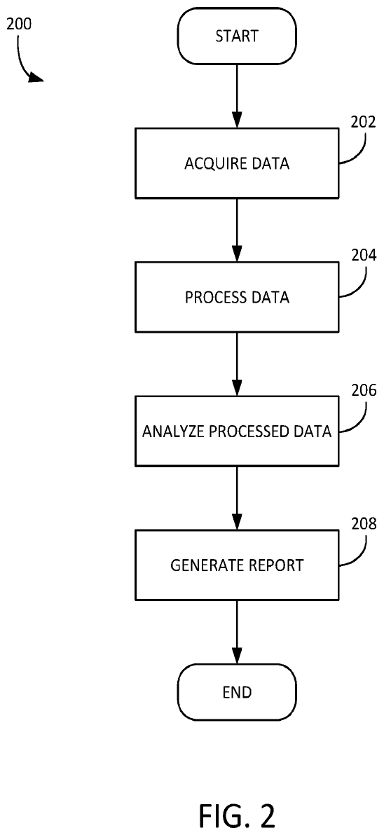

Referring to FIG. 2, a process 200 in accordance with the present disclosure, which may use a system 100 such as described with respect to FIG. 1 for in vivo or ex vivo analysis, may begin at process block 202 with the acquisition of either “sequential” or “cumulative” data. As will be explained, the data can be acquired as accumulation of time-domain (TD) or, expressed another way, forms quasi time domain data (QTD). Additionally, traditional, intensity data may be acquired. As will be described, such intensity data may be used with and/or may be reported in addition to or combined with lifetime data. Thus, lifetime data is acquired and, optionally, intensity data can be acquired.

In sequential TD acquisition, time resolved (or time-series) fluorescence data are collected for multiple time points with fixed ‘gate widths’ (or acquisition window/exposure) and fit to exponential decays:

$${y}^{TD} ( t ) = {a}_{0} {e}^{- \frac{t}{τ}} ; \qquad ( 1 )$$

where y^{TD} refers to the data as a function of time ‘f’, a₀ is a decay amplitude, which is related to the fluorophore concentration, quantum yield and other experimental scaling constants, and ‘τ’ refers to the fluorescence lifetime.

However, in accordance with one aspect of the present disclosure, TD fluorescence data are acquired cumulatively from a chosen time origin to an end point or to multiple time points. This creates a varying gatewidth/time window/exposure, which is represented mathematically as:

$${y}^{QTD} ( t ) = {\int }_{0}^{t} y ( {t}^{′} ) {dt}^{′} ; \qquad ( 2 )$$

where QTD refers to “quasi time domain” acquisition. Using equation (1) in equation (2), it can be shown that:

$${y}^{QTD} ( t ) = {a}_{0} {\int }_{0}^{t} {e}^{- \frac{{t}^{′}}{τ}} {dt}^{′} = {a}_{0} τ ( 1 - {e}^{- \frac{t}{τ}} ) . \qquad ( 3 )$$

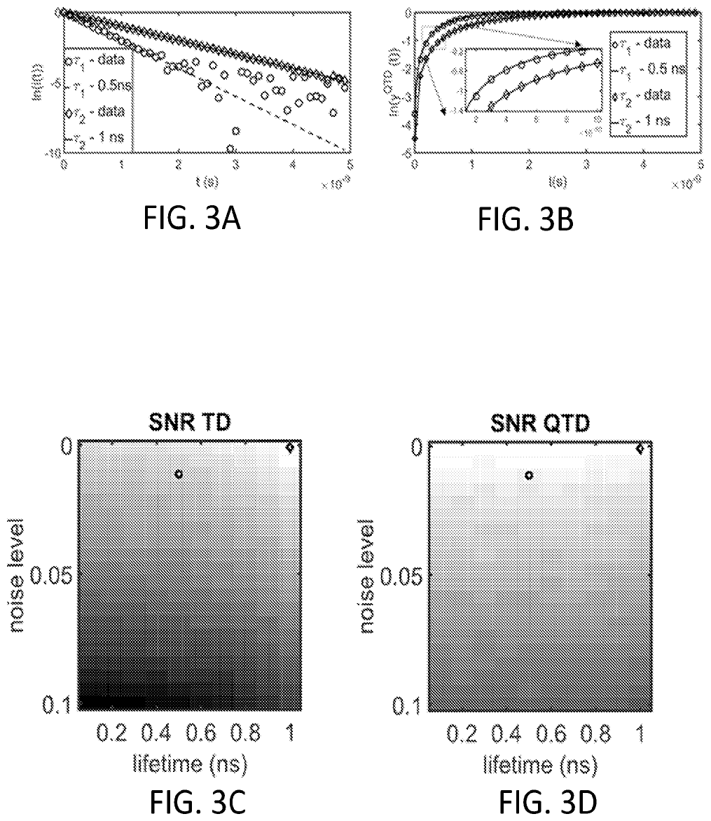

Thus, the QTD data is fit to the function, τ(1−e^{-t/τ}), rather than e^{-t/τ}. While the two methods are mathematically equivalent, a key difference in the experimental aspect is that the QTD method can provide significantly improved signal to noise ratio (SNR) compared to the standard TD method. That is, the QTD method results in overall higher signal counts and higher SNR for all time points in a short noise limited system because lower count signals at later time points are cumulatively combined with all the earlier, higher count signals. This principle is demonstrated in the FIGS. 3A-3D for two lifetimes, T₁=0.5 ns and T₂=1 ns. In particular, FIG. 3A shows TD fluorescence decays for two different fluorophores with lifetimes of 71=0.5 ns (circles) and 12=1 ns (diamonds), with the corresponding non-linear fits to the data using the function e^{-t/τ}. (solid and dashed lines). FIG. 3B shows the quasi-time domain (QTD) for the same two lifetimes in FIG. 3A, shown with the non-linear fits to the function τ(1−e^{-t/τ}). The SNR improvement with QTD is clear especially for the shorter lifetime of 0.5 ns. FIG. 3C shows the SNR for a range of lifetimes and noise levels using the standard TD. Finally, FIG. 3D shows the SNR for varying lifetimes and noise levels using the QTD showing significant improvement in SNR compared to standard TD for the entire range of lifetimes and noise levels simulated. In FIGS. 3C and 3D, the circle and diamond indicate the parameters for which the temporal responses in FIGS. 3A and 3B are shown. The improved SNR in QTD allows the use of a fewer number of temporal data points, thereby resulting in improved imaging speed.

In one aspect of the QTD data acquired, the data and the fluorescence intensity could be used to compute the lifetime map directly. From equation (3):

$${y}^{QTD} ( t ) = {a}_{0} τ ( 1 - {e}^{- \frac{t}{τ}} ) = {y}^{QTD} ( ∞ ) ( 1 - {e}^{- \frac{t}{τ}} ) . \qquad ( 4 )$$

Here, γ^{QTD}(∞)=∫₀^{∞}α₀e^{-t/τ}dt=a₀τ, which is the continuous wave (CW) intensity data. For a suitable gate width T₁, the above equation can be solved for the lifetime to obtain:

$$τ = - \frac{{T}_{1}}{log ⌈ 1 - \frac{{y}^{QTD} ( {T}_{1} )}{{y}^{QTD} ( ∞ )} ⌉} . \qquad ( 5 )$$

Since the SNR of γ^{QTD}(∞)≥γ^{QTD}(T₁)>γ_{τD}, the single gate width and CW data can give a better estimate of the lifetime maps, which otherwise require multiple time delays in the conventional time domain methods due to the inherent noise in the data. This single-time gate approach drastically decreases both the acquisition and computation time, leading toward real-time lifetime maps.

In another aspect of QTD data, it can be proved theoretically that the uncertainty in the QTD data is less than that of the TD data. Based on equation (4), the relation connecting QTD and TD may be written as:

$${y}^{TD} ( t ) = {a}_{0} ( 1 - \frac{{y}^{QTD} ( t )}{{y}^{QTD} ( ∞ )} ) . \qquad ( 6 )$$

The uncertainties of equation (6), can be written as,

$${\text{?}}_{\text{?}}^{\text{?}} = {\text{?}}_{\text{?}}^{\text{?}} ( \frac{{\text{?}}_{\text{?}}^{\text{?}}}{{\text{?}}^{\text{?}} ( \text{?} )} \text{?} \frac{{\text{?}}_{\text{?}}^{\text{?}} ( \text{?} )}{{\text{?}}^{\text{?}} ( \text{?} )} ) .$$

$$\text{?} \text{indicates text missing or illegible when filed}$$

Here, the σ_{TD}, σ_{QTD}, σ_{∞} are the uncertainties in the TD, QTD and CW data, respectively. From this, it can be proved that σ_{QTD}<σ_{TD}. This shows that uncertainties in QTD is lesser than in TD, thus leading to better estimation of the lifetime maps.

Referring to FIGS. 3A and 3B, the TD fluorescence data from sequential or cumulative acquisition can be linearized based on logarithmic or series expansion or decomposition schemes. The resulting linearized data can be fit for the lifetimes using any linear least squares based approaches.

Referring again to FIG. 2, at process block 204, the lifetime data and any optional intensity data is processed for analysis. In one non-limiting example, the acquired data can be analyzed a variety of different ways. In one non-limiting example, such analysis may use exponential functions or decay amplitude maps, wherein the data is resolved into exponential functions with known lifetimes for the tumor and normal tissue based on prior characterization as described earlier:

$${y}^{TD} ( t ) = {a}_{tumor} exp ( - \frac{t}{{τ}_{tumor}} ) + {a}_{normal} exp ( - \frac{t}{{τ}_{normal}} ) . \qquad ( 7 )$$

In another non-limiting example, the decay amplitudes αₜᵤₘₒᵣ and αₙₒᵣₘₐₗ can be recovered from the QTD data with two-time gates and CW intensity data, if the tumor and normal lifetimes τₜᵤₘₒᵣ and τₙₒᵣₘₐₗ are known a priori. This is done as follows.

Based on equation (3), the QTD data for a given time gate ‘T’ can be written in bi-exponential form as:

$${y}^{QTD} ( T ) = {a}_{tumor} {τ}_{tumor} ( 1 - {e}^{- \frac{T}{{τ}_{tumor}}} ) + {a}_{normal} {τ}_{normal} ( 1 - {e}^{- \frac{T}{{τ}_{normal}}} ) . \qquad ( 8 )$$

The term αₜᵤₘₒᵣτₜᵤₘₒᵣ+αₙₒᵣₘₐₗτₙₒᵣₘₐₗ is just the CW intensity data, i.e., γ^{(TD)}_{(∞)}. Replacing it in equation (8) gives:

$${y}^{QTD} ( ∞ ) - {y}^{QTD} ( T ) = {a}_{tumor} {τ}_{tumor} {e}^{- \frac{T}{{τ}_{tumor}}} + {a}_{normal} {τ}_{normal} {e}^{- \frac{T}{{τ}_{normal}}} . \qquad ( 9 )$$

The above equation can be solved for the decay amplitudes αₜᵤₘₒᵣ and αₙₒᵣₘₐₗ using two gate widths T₁ and T₂ using a linear matrix formulation as follows:

$$[ \begin{matrix}{τ}_{tumor} {e}^{- \frac{{T}_{1}}{{τ}_{tumor}}} & {τ}_{tumor} {e}^{- \frac{{T}_{2}}{{τ}_{tumor}}} \\ {τ}_{normal} {e}^{- \frac{{T}_{1}}{{τ}_{normal}}} & {τ}_{normal} {e}^{- \frac{{T}_{2}}{{τ}_{normal}}}\end{matrix} ] [ \begin{matrix}{a}_{tumor} \\ {a}_{normal}\end{matrix} ] = [ \begin{matrix}{t}^{QTD} ( ∞ ) - {y}^{QTD} ( {T}_{1} ) \\ {t}^{QTD} ( ∞ ) - {y}^{QTD} ( {T}_{2} )\end{matrix} ] . \qquad ( 10 )$$

The amplitude coefficients αₜᵤₘₒᵣ and αₙₒᵣₘₐₗ are next recovered using a linear fit to the raw data. These amplitude map, αₜᵤₘₒᵣ, corresponding to the tumor lifetime can be displayed on the sample surface, as will be further described. The advantage of such a linear fit is a dramatic increase in acquisition speed.

Regardless of the particular analysis process, the overall processing of the data can be helpful to enhance tumor contrast, without requiring specific chemicals design to target cancer. The design of chemical probes with cancer specificity has been challenging and has not been successful to date since cancer-specific markers are also expressed in normal tissue. On the other hand, the present disclosure can use fluorescence lifetime distinctions between the dyes taken up by cancer cells versus the lifetimes of the dyes in healthy tissue, allowing dramatic sensitivity and specificity enhancement compared to traditional fluorescence intensity based detection. In this regard, a threshold may be used that distinguishes cancerous tissue. For example, images may be reconstructed, which can then be analyzed against one or more thresholds at process block 206.

In one non-limiting example, the threshold may be selected to delineate tumor from normal tissue. This threshold may be selected to make discrimination applicable to multiple patients, at least for a given type of cancer. In other words, different types of primary and metastatic cancer (e.g., oral, brain, skin, breast, liver, melanomas, and sarcomas) may be discriminated using a different FLT threshold, but a given cancer type can be discerned using the FLT threshold across multiple patients, independent of the measurement system or other variables.

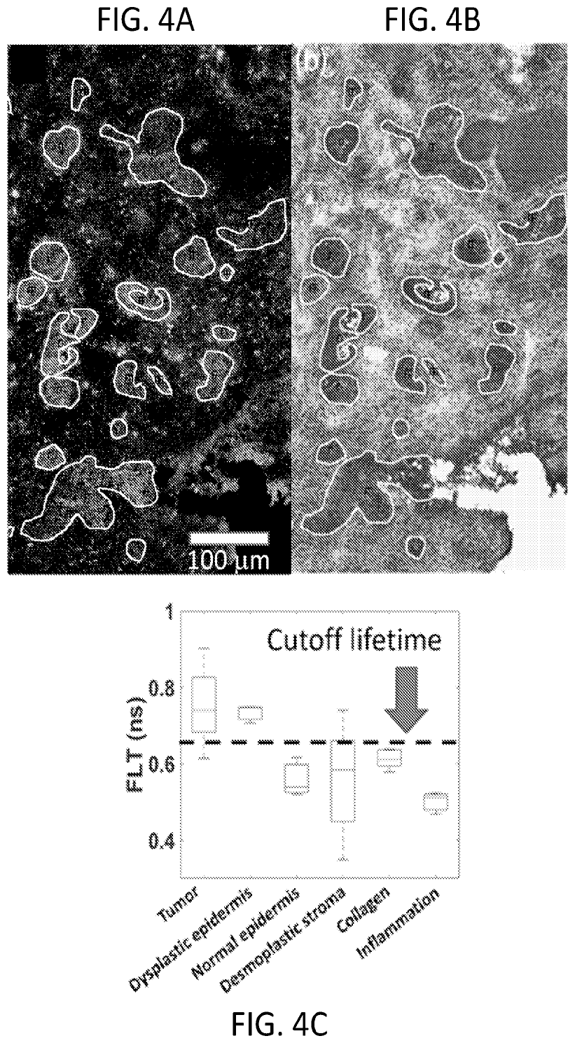

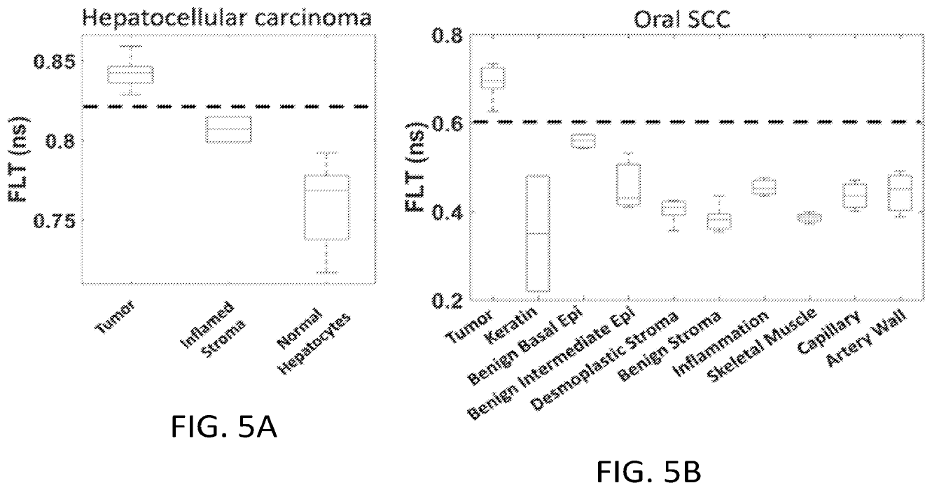

For example, referring to FIGS. 4A-4C, FLT threshold selection is illustrated relative to cutaneous skin cancer. In particular, the FLTs for various tissue types can be initially tabulated from fluorescence lifetime microscopy (FLIM) measurements of tissue sections, as illustrated in FIG. 4A, and co-registered with the histology images, as illustrated in FIG. 4B, from patients infused with the fluorescent dye, which in this case was ICG. These microscopic lifetime values indicate tumor-specific FLTs as well as the FLTs for various types of normal tissue, and can be tabulated in a box plot, such as illustrated in FIG. 4C. Receiver operating characteristic (ROC, plot of sensitivity vs specificity vs varying threshold) can be calculated for tumor vs normal tissue classification. The FLT threshold can then be determined as the FLT value that provides the desired accuracy, for example, calculated as the area under the ROC curve, for tumor versus normal classification. As a further example, FIGS. 5A and 5B, show similar box plots for liver (hepatocellular carcinoma) and (oral squamous cell carcinoma) cancers, indicating their associated threshold lifetimes. Thus, the present disclosure demonstrates that the systems and methods provided herein extend across anatomy and tumor type.

Although our preliminary data indicate a particular range of values for the threshold lifetimes, these values can be updated with further clinical studies and multiple patients injected with a particular fluorescent dye. The lifetime threshold for each cancer type could be determined across various conditions including but not limited to time and dosage of injection and pre-treatment status (such as radiation and chemotherapy).

Referring again to FIG. 2, the result of the analysis performed at process block 206 is then used to generate a report at process block 208. In a surgical setting, the FLT threshold for a particular cancer can be used to define a tumor/normal boundary that may be included in the report at process block 208. Furthermore, the tumor/normal boundary may be overlaid on an anatomical image or projected onto the surgical bed to guide the operating surgeon on where to begin resection and how much tissue to cut. If acquired/included, intensity data can be provided in the report. In this way, the intensity data can serve as a reference for clinicians or further validation of the reporting using lifetime data. Furthermore, the intensity data can be reported to guide tumor location in real-time or near real-time and/or to guide collection of the lifetime data and images. Thus, the report generated at process block 208 may take a variety of forms.

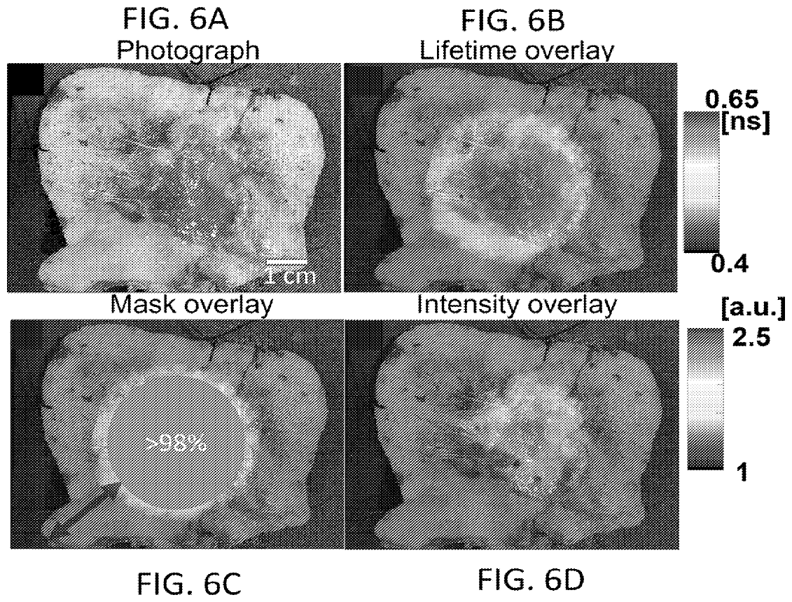

Referring now to FIGS. 6A-6D, one non-limiting example of a report including an overlay is shown. In particular, FIG. 6A is an anatomical photograph of a specimen removed from a patient with cutaneous (skin) squamous cell carcinoma, injected with ICG 24 hours prior to surgery. Then, FIG. 6B shows a color-coded FLT overly, which can be readily understood by a clinician or translated into a masked region, as shown in FIG. 6C, which indicates the tumor region with an accuracy of >98%. Such a mask can be readily created in accordance with the present disclosure, for example, by selecting a desired threshold value, such as described above. This stands in stark contrast to an overlay of fluorescent intensity shown in FIG. 6D, which would lead the clinician to resect far too little tissue and, thereby, lead to the need for multiple, serial operations or substantial treatments.

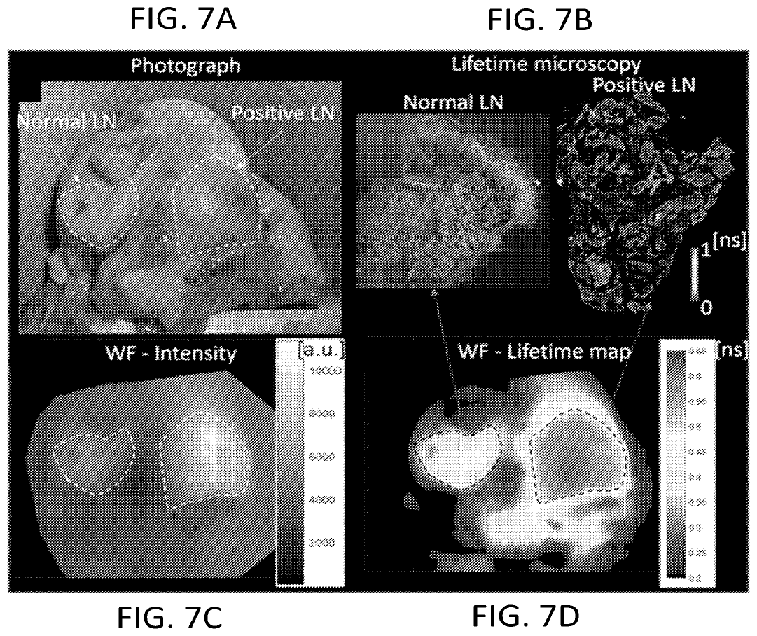

The concept of a FLT threshold (or cutoff) can also be applied to delineate positive from negative lymph nodes during surgeries and neck dissections. Clinical data demonstrating this application is shown in FIGS. 7A-7D. In particular, FIG. 7A shows the color photograph of the specimen resected from a patient with SCC, undergoing radical neck dissection. Two lymph nodes (LN) were identified in the specimen (white dashed lines), only one of which was later confirmed histologically to be positive for tumor. FIG. 7B shows microscopic FLT images from small sections within the positive and negative LNs, showing the significantly longer lifetime in the positive LN. FIGS. 7C and 7D show the wide-field fluorescence intensity and FLT maps of this specimen indicating that the FLT values within the positive LN are significantly and uniformly longer than the FLTs of the negative LN, while the intensity shows significant heterogeneity. Moreover, intensity is a relative quantity and is expressed in arbitrary units, making the distinction between tumor and normal tissue more ambiguous, especially across multiple patients or imaging systems. FLT however is an absolute quantity that can be used to define an absolute

In a diagnostic setting, e.g., breast cancer screening, the threshold lifetime can be directly applied to TD fluorescence data collected with the intact breast, provided the subject has been infused with ICG (or another dye, for example an EGFR targeted dye) prior to the imaging. The threshold lifetime can be used to diagnose the presence of malignant versus benign tumors non-invasively.

Thus, the reports generated may take a variety of different forms. For example, data acquisition at process block 202 may simultaneously acquire both traditional fluorescence intensity (also termed “continuous wave” (CW) fluorescence) and FLT data for display during surgery or screening. In this case, the system can simultaneously display both the CW fluorescence that is shown by standard cameras, and FLT images in the reports, which can be generated in real time. This is useful in a variety of clinical settings, where ICG fluorescence accumulation can be directly revealed by the intensity image, which can be collected in real-time, thereby enabling the surgeon quick navigation to the region of interest before precisely localizing the tumor using the fluorescence lifetime maps. Thus, with reference to FIGS. 6A-6D, the report may provide the clinician with images such as illustrated in FIG. 6D in real time, which are then juxtaposed or replaced by a lifetime overlay (FIG. 6B) or a mask overlay (FIG. 6C) as the FLT data is acquired and processed.

The systems and methods described herein can be incorporated under a wide range of implementations, depending on the specific clinical application. For example, for open surgeries, a wide-field, time-gated, intensified camera or array detectors can be used either directly or with fiber bundles for collecting light from the surgical bed. In some scenarios the resected specimens can also be visualized using the same camera or detector array to identify the presence of tumor at the surgical boundary and to inform pathology processing.

For imaging of deep organs in the body such as the lungs or liver, endoscopes or laparoscopes can be used for minimally invasive surgeries. In such scenarios pulsed light can be delivered via fibers to the light input port of the laparoscope/endoscope, and the fluorescence emitted from the detection port of the scope can be coupled to either the intensified charge coupled device (ICCD) camera or to one or more photomultiplier tube (PMT) detectors. In the case of ICCD camera detection is performed via a time gating mechanism. In the case of PMTs detectors, time resolved data can be acquired using time correlated single photon counting (TCSPC) schemes. Confocal imaging techniques could also be used in conjunction with endoscopes to obtain micron level resolution in both in situ during surgery and in ex vivo tissue.

Illumination can be either in the form of uniform wide-field illumination or in the form of spatially patterned illumination. Pattered illumination is useful in obtaining accurate FLT maps in the presence of tissue scattering as described in U.S. Pat. No. 9,921,156, which is incorporated herein by reference in its entirety.

### Examples

We have observed, using multiple clinical and animal studies, that immediately following, and up to 96 hours after intravenous injection of indocyanine green (ICG) and EGFR-targeted fluorescent probes, the fluorescence lifetime of tumors is significantly longer than the lifetime of surrounding normal tissue. This difference in lifetime between ICG in tumor and normal tissue allows the separation of tumors from background with more than 98% accuracy, which is significantly better than current methods that employ intensity-based fluorescence imaging that can result in low accuracy of 50%. It is noted that the 98% accuracy could be further improved with further instrumentation design focused on FLT imaging.

Using high-resolution microscopic imaging and cell cultures, studies have also confirmed that the increased lifetime of ICG or other near infrared dyes arises from dye localized within the tumor cells (and not from the dye retained in the tumor environment outside the tumor cells). Therefore, using appropriate devices such as endoscopes, the systems and methods of the present disclosure can be used to detect microscopic residual cancer in the body.

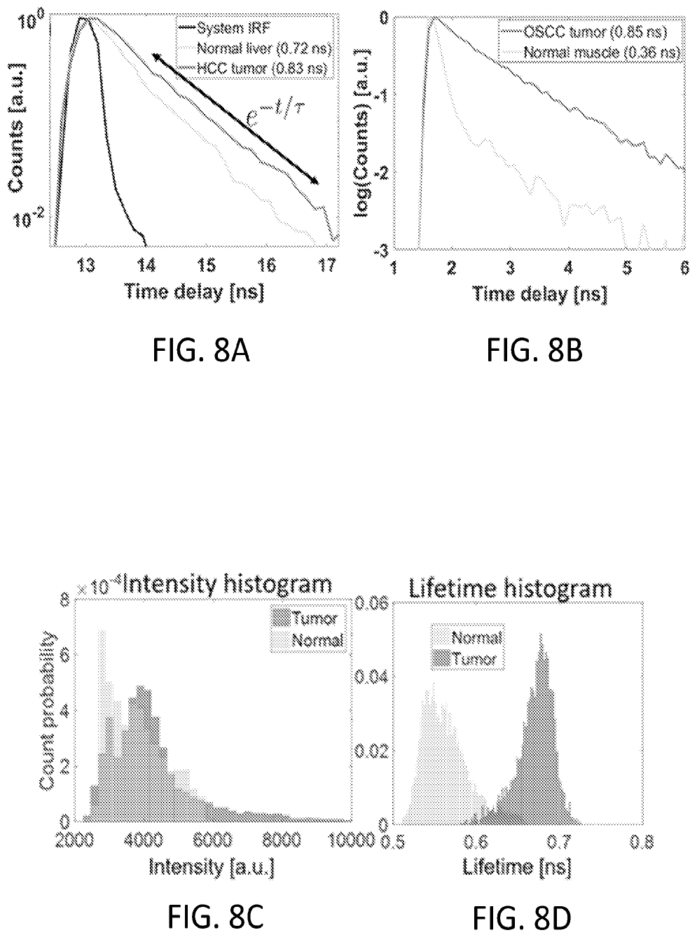

An IRB-approved, pilot clinical study was performed using fresh specimens from 25 patients scheduled for surgery of metastatic colorectal cancer (mCRC), hepatocellular carcinoma (HCC), and oral squamous cell carcinoma (OSCC), cutaneous squamous cell carcinoma (CSCC) or bone and soft tissue sarcomas. Patients were injected with ICG between 2 hours and 72 hours prior to surgery and the resected specimens were imaged in the frozen section lab, with a prototype time domain (TD) fluorescence specimen imaging system, such as described above and illustrated in FIG. 1. FIG. 8A shows the time domain (TD) fluorescence signals measured from liver HCC. The decay portion of the TD signal (arrow in FIG. 8A) is fit to a single exponential decay to obtain the FLT maps, τ(r), where r is the pixel location. Similarly, FIG. 8B shows TD fluorescence signals measured from oral SCC cancer surgical specimens freshly resected from patients systemically injected with ICG 24 to 48 hours prior to surgery. In both cases, the FLT (t) (even when calculated from the decay of TD fluorescence data by fitting to single exponentials, e^{-t/τ}, where t is the time delay) is significantly longer in tumors compared to normal tissue.

Data acquired from a liver specimen with an HCC tumor was also used for additional comparison and analysis. Despite the high non-specific ICG uptake, the FLTs within the tumor were significantly longer than the FLTs in surrounding tissue with minimal overlap. FIG. 8C shows a histogram of instantaneous intensity and shows that tumor and normal tissue are highly overlapping. On the other hand, histograms generated from FLT data, as illustrated in FIG. 8D, shows minimal overlap.

Receiver operating characteristic (ROC) curves were generated by plotting sensitivity versus specificity for varying intensity and FLT thresholds. Sensitivity was defined by the number of pixels within tumor with intensity or FLT above the threshold divided by the total number of pixels within the tumor. False positive rate was defined as the number of pixels outside tumor above the threshold intensity or FLT threshold divided by the total number of pixels outside tumor. The accuracy was calculated as the area under the curve (AUC) and was 98% for FLT-based tumor/normal classification, compared to 40% for intensity-based tumor/normal classification.

It is noteworthy that this held true even in a patient with significant cirrhosis in the liver, suggesting that any non-specific uptake due to cirrhosis did not impact the tumor delineation accuracy using FLT data. A ratiometric fluorescence/reflectance analysis did not improve the accuracy of intensity-based classification, likely because the low tumor contrast is due to non-specific dye accumulation, rather than tissue absorption or scattering variations. FLT imaging microscopy (FLIM) (Stellaris 8, Leica) of lymph nodes with mCRC tumor infiltrates resected from another patient, indicated excellent agreement of FLT-based classification with histology and the ability of FLT to delineate sub-millimeter tumors, formed of microscopic cancer cell nests with longer FLT than the tumor stroma. For all tumor types studied, the tumor FLT was significantly longer than the FLT of normal tissue, providing >97% classification accuracy. These results demonstrate the ability to use the systems and methods of the present disclosure for margin guidance in liver cancers, where standard intensity-based fluorescence methods are clearly inadequate.

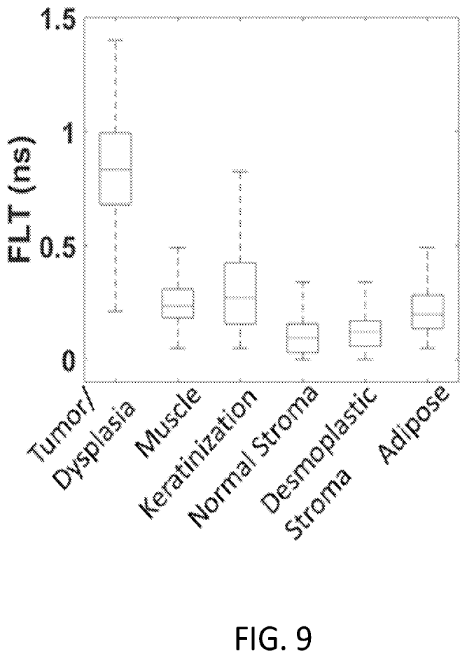

FIG. 9 is a box-and-whisker plot of FLTs from 13 different subjects, indicating that the mean tumor FLTs across multiple subjects exhibit ˜5% variation, and are significantly longer than the FLTs of tissue autofluorescence or nonspecific ICG in normal/healthy tissue. Therefore, this again demonstrates that it is viable to use a “threshold” FLT for tumor versus normal classification that is consistent across multiple subjects for a given tumor type.

In accordance with one particular and non-limiting study, for widefield time-domain imaging, a system akin that of FIG. 1 was used that included a Supercontinuum laser and tunable filter (EXR-20, SuperK Varia, NKT Photonics, 80 MHz repetition rate; 400-850 nm tuning range) providing 770±30 nm excitation, a multimode fiber (Thorlabs, Newton, New Jersey, United States) delivering light to the sample, and a gated intensified CCD (LaVision, Picostar, 500 V gain, 0.1 to 1 second integration time, 150 ps steps, 256×344 pixels after 4×4 hardware binning). A digital micromirror device was used to expand the output of the optical fiber and delivered to the surface of the animal. The average total power across the illumination area (approximately 6×8-cm) was 10 to 20 mW. Fluorescence was collected in reflectance mode using an 835/70-nm band-pass filter. TD fluorescence imaging was performed with a gate width of 600 ps and 150 ps steps for a total duration of approximately 6 ns per laser duty cycle of 12.5 ns. In vivo animal imaging was performed 48 hours after intravenous injection of panitumumab-IRDye800CW (150 μl, 1 mg/ml). Animals were sacrificed after imaging and tumors were immediately frozen in optimal cutting temperature (OCT) compound for fluorescence lifetime imaging microscopy (FLIM), histology and immunohistochemical (IHC) staining.

For multispectral imaging, paraffin blocks of ex vivo clinical specimens were imaged in an IVIS Spectrum CT imaging system (PerkinElmer, Waltham, Massachusetts, United States) using a 710 nm excitation and 760-840 nm emission wavelengths. Camera integration time was automatically adjusted during image acquisition and the Living Image software was used to extract the fluorescence images normalized to integration time. True fluorescence emission spectra of panitumumab-IRDye800CW and tissue autofluorescence were used as basis functions to perform a linear deconvolution of the multispectral images and the amplitudes of panitumumab-IRDye800CW and tissue autofluorescence were extracted.

Also, a STELLARIS 8 FALCON (Leica, Germany) FLIM system was used for NIR FLIM of 10-μm thin tissue sections (murine tumors and clinical specimens). Imaging was performed using 730 nm excitation with 750 nm notch filter and detected with a HyD R detector operating within 770-850 nm range. A 10×, 0.4 NA objective was used for image collection and digital images with 512×512 pixels (2.275 μm/pixel), 4 line repetitions and 4 line averages were obtained. TD data was collected using time-correlated single photon counting.

With these systems, a variety of studies were performed. Conjugation of panitumumab-IRDye800CW was performed under cGMP conditions. Briefly, Panitumumab (Vectibix; Amgen, Thousand Oaks, California, United States) was concentrated, and pH adjusted by buffer exchange to a 10 mg/mL solution in 50 mmol/L potassium phosphate, pH 8.5. IRDye800CW (IRDye800CW-N-hydroxysuccinimide ester, LI-COR Biosciences, Lincoln, Nebraska, United States) was conjugated to Panitumumab for 2 hours at 200 C in the dark, at a molar ratio of 2.3:1. After filtration with desalting columns (Pierce Biotechnology, Rockford, Illinois, United States) to remove unconjugated dye and buffer exchange to PBS, pH 7, the final protein concentration was adjusted to 2 mg/ml. The product was sterilized by filtration and placed into single-use vials and stored at 40 C until used.

EGFR overexpressing cell line MDA-MB-231 and EGFR negative cell line MCF7 were purchased from ATCC and cultured in high glucose DMEM supplemented with 10% fetal bovine serum (FBS) and 1% penicillin-streptomycin (Life Technologies). An oral cancer cell line was maintained in RPMI culture media supplemented with 10% FBS and 1% penicillin-streptomycin. Cells were harvested at 80% confluency for tumor induction.

For in vitro experiments, FaDu cells were plated at 0.2×106 cells per well in a 12-well plate containing poly-D-lysine coated glass coverslips and were allowed to adhere to the coverslips for 24 hours. Cells were then incubated with panitumumab-IRDye800CW (100 μg), IgG-IRDye800CW (100 μg) or PBS (pH 7.4) for 2 hours at 370 C. After probe incubation, cells were fixed in 4% para-formaldehyde (PFA) and mounted with ProLong Gold Antifade medium (ThermoFisher Scientific, Waltham, Massachusetts, United States) for confocal FLIM.

With respect to patients, all patients were given informed consent, and the study protocol was approved by IRB and the FDA. The study was performed in accordance with the Helsinki Declaration of 1975 and its amendments, FDA's ICH-GCP guidelines, and the laws and regulations of the United States. The manufacturing process of panitumumab-IRDye800CW by the Fredrick National Labs is described previously. Consented patients meeting study criteria were admitted to the infusion center for panitumumab-IRDye800CW administration. Panitumumab-IRDye800CW was systemically administered at a dose of 0.6 mg/kg 48 hours prior to surgery. Ex vivo OSCC tissue from patients systemically injected with panitumumab-IRDye800CW were formalin fixed, dissected, and paraffin embedded. Paraffin embedded tissue blocks were then moved to the TD imaging study.

All animal studies were approved by the Institutional Animal Care and Use Committee in accordance with the animal welfare guidelines at Massachusetts General Hospital. Seven (4- to 6-week-old) female nu/nu mice were purchased from Charles River Laboratories Inc and were housed at the animal facility in Massachusetts General Hospital (Boston, MA). Animals were quarantined for 1 week and kept in a normal diet with 12-hour light and dark cycle. After 1 week, animals were anesthetized with 3% isoflurane and subcutaneously injected with 2×106 MDA-MB-231 (n=5, EGFR overexpressing) or MCF7 (n=2, EGFR negative) cells in 1:1 PBS:Matrigel mixture. Mice with MCF7 cells were also implanted with a slow release estrogen pellet to expedite tumor growth. Tumors were measured once every two days until they reached 5- to 10-mm diameter in one dimension.

For histopathology and immunohistochemistry, OSCC tumors with surrounding normal tissue were fixed in 10% formalin, embedded in paraffin, sectioned (10-μm thickness), and stained with hematoxylin and eosin (H&E) or processed for IHC. For IHC, 10-μm thick paraffin-embedded tissue sections were dewaxed in xylene and rehydrated in decreasing concentration of alcohol. Antigen retrieval was performed with EDTA (pH 9.0) at sub-boiling temperature for 15 minutes. Tissue sections were incubated in 1:50 dilution of anti-EGFR antibody (Cat #4267, Cell Signaling Tech.) overnight at 4° C. Secondary antibody was applied for 30 minutes at 37° C. and slides were developed with DAKO HRP-compatible DAB (Cat #SF-4100, Vector Laboratories, Burlingame, California, United States) and counterstained with Harris Hematoxylin. Images of H&E- and IHC-stained tissue sections were obtained using an inverted Keyence BZ-X810 microscope (Keyence, Itasca, Illinois, United States). A Plan Apo 10×, 0.45 NA air objective (Nikon, Tokyo, Japan) and a monochrome CCD (colorized with LC filter) were used to capture images. Histology images were graded by two experienced pathologists.

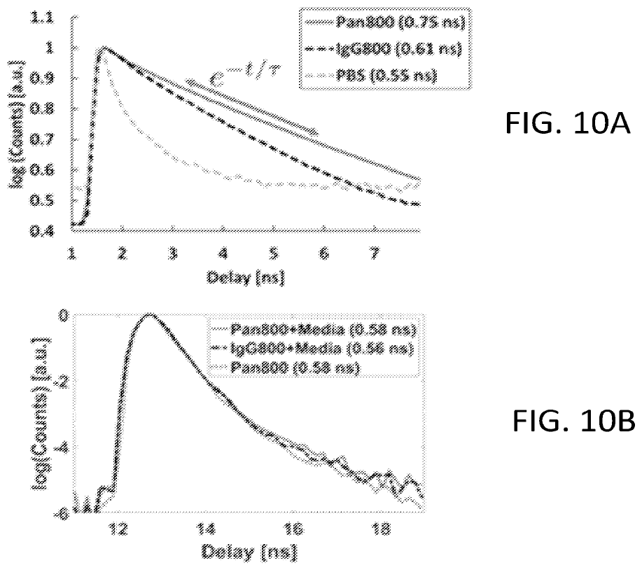

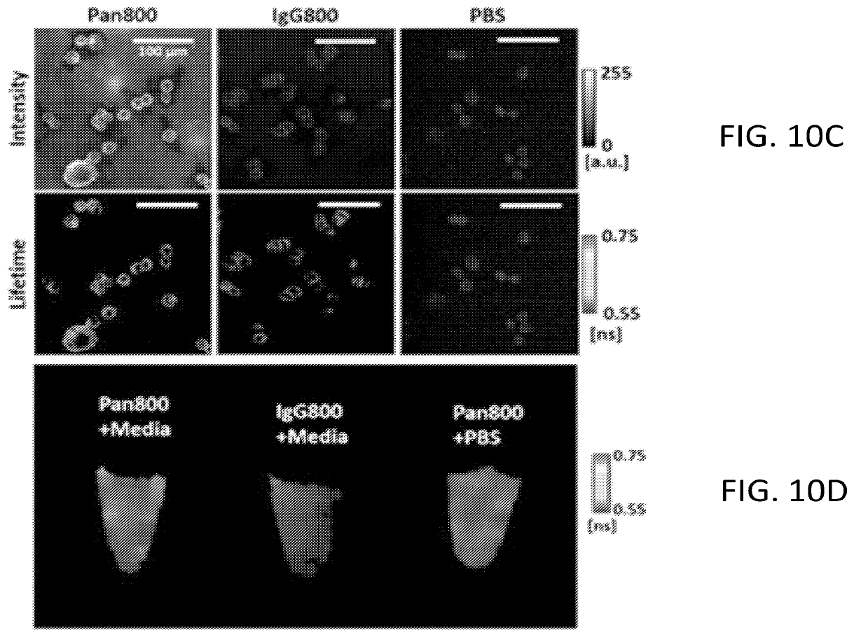

For the widefield TD data analysis, TD fluorescence images were analyzed in MATLAB (MathWorks, Natick, Massachusetts, United States) using a custom software. As illustrated in FIGS. 10A and 10A, TD data from individual pixels were plotted as time gate versus log (counts) and the FLT was obtained by fitting the decay portion of TD fluorescence profiles to a single exponential function, e−t/τ(r), where r denotes pixel location and τ(r) constitutes a lifetime map. More particularly, representative fluorescence decay curves of panitumumab-IRDye800CW (gray solid), IgG-IRDye800CW (black dashed) and PBS (gray dashed) in cancer cells are shown in FIG. 10A. Furthermore, FIG. 10A shows representative fluorescence decay curves of panitumumab-IRDye800CW (gray solid) and IgG-IRDye800CW (black dashed) in culture media, and the stock solution of panitumumab-IRDye800CW in PBS (gray dotted).

Histology images were co-registered with fluorescence intensity and FLT maps. Histologically confirmed regions of interest (ROIs) for tumor and normal tissue were then mapped onto the co-registered fluorescence intensity and FLT images. The intensities and FLTs from pixels enclosed by the ROIs were used to calculate probability distributions for pixels as normal or tumor. Receiver operating characteristic (ROC) curves were obtained by varying the threshold for intensity and FLT and computing sensitivity and specificity. Sensitivity is denoted as the number of pixels within the tumor ROI above the intensity or FLT threshold, divided by the total number of pixels within the tumor ROI. Specificity was calculated as the number of pixels within the normal ROI below the threshold divided by the total number of pixels within the normal ROI.

For the FLIM and IHC image analysis, the FALCON/FLIM software was used to collect and analyze the FLIM data. Lifetime values at each pixel location was calculated by using a single exponential fitting of the fluorescence decay curves. Large area stitched FLIM and IHC images from each tissue slices were first co-registered using a custom MATLAB code. Images were then divided into multiple regions of interest (ROIs) with a 300×300 pixel size. ROIs with less than 10% pixels represented by tissue were excluded from further analysis. IHC image ROIs were analyzed by color deconvolution using the IHC Tool Box in ImageJ (NIH, Version 1.48u) to extract EGFR positive pixels within each ROI. EGFR expression level in the ROIs were represented as percent of EGFR positive pixels. Corresponding FLIM image ROIs were analyzed by averaging FLT values above 0.3 ns. EGFR expression and average FLT values of each pair of IHC and FLIM ROIs were compared using a scatter plot and correlation coefficient.

Statistical analysis was carried out using Mann-Whitney U test (two-tailed) to estimate p values for bar graphs. P values less than 0.05 were considered significant: *, P<0.05, and **, P<0.01. The experiments were not randomized, and the investigators were not blinded to allocation during experiments and outcome assessment. Results are presented as mean±standard deviation.

### Results

In vitro measurements establish that the cellular specificity of FLT increases in a high EGFR expressing head & neck cancer cell line, using fluorescence lifetime imaging microscopy (FLIM). For example, FIG. 10C shows microscopic fluorescence intensity and FLT maps of FaDu cells, incubated with panitumumab-IRDye800CW (left), IgG-IRDye800CW (center) or PBS (right). Cells treated with panitumumab-IRDye800CW showed higher probe uptake and longer FLT values (˜0.75 ns) compared to the cells incubated with IgG-IRDye800CW, which showed low uptake and a short FLT (˜0.61 ns). Cells incubated with PBS showed basal level autofluorescence and an average FLT of 0.55 ns under the same experimental conditions. Representative fluorescence decay profiles, as provided in FIG. 10A showed the longest decay time for panitumumab-IRDye800CW, followed by IgG-IRDye800CW and PBS in cells. Panitumumab-IRDye800CW and IgG-IRDye800CW in culture media collected from in vitro experiments showed, as illustrated in FIG. 10D, short FLTs of 0.58 ns and 0.56 ns respectively, which were comparable to the FLT of the stock solution of panitumumab-IRDye800CW in PBS (0.58 ns), indicating that environmental effects due to culture conditions did not cause the observed FLT increase of panitumumab-IRDye800CW in cancer cells. FIG. 10A shows representative fluorescence decay profiles of panitumumab-IRDye800CW and IgG-IRDye800CW in culture media, which were indistinguishable from the decay profile of panitumumab-IRDye800CW in PBS. These in vitro experiments indicate an EGFR specific uptake and FLT enhancement of panitumumab-IRDye800CW in cancer cells.

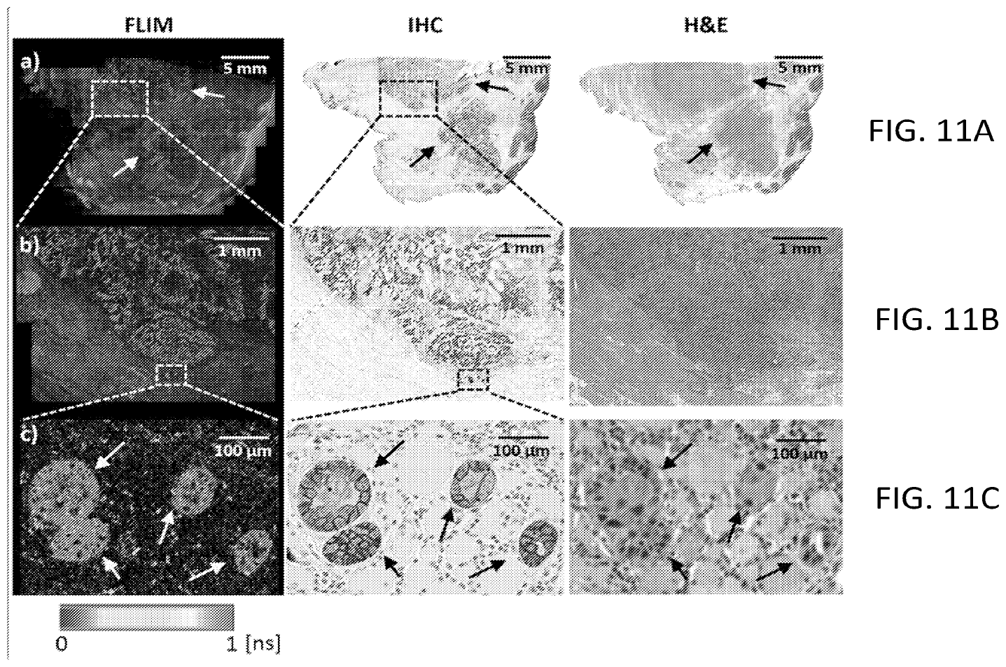

Also, a retrospective imaging study was performed using tissue specimens from glossectomy of the lateral tongue of OSCC patients who received a systemic injection of panitumumab-IRDye800CW, 48 hours prior to surgery. Specifically, FIGS. 11A-11C show FLIM (left), EGFR IHC (center) and H&E stained histology (right) images from a representative specimen, illustrating the longer FLT in OSCC tumors corresponding to the higher EGFR expression in the tumor region. More particularly, FIG. 11A shows a large field of view region of interest (ROI). Long FLT (red) is observed in two EGFR overexpressing tumor clusters indicated by the arrows. Low EGFR expressing normal tissue surrounding the tumor showed shorter FLTs (green/blue) consistent with the FLTs of nonspecific panitumumab-IRDye800CW and tissue autofluorescence. Also, FIGS. 11B and 11C show higher magnification regions of interest (ROIs, shown as dashed boxes) from FIG. 11A and FIG. 11B, respectively. The FLIM images show long FLTs spatially colocalized within high EGFR expressing tumor cells and the tumor specificity of FLT enhancement can be observed in individual OSCC cell clusters down to single cell resolution (FIG. 11C, arrows).

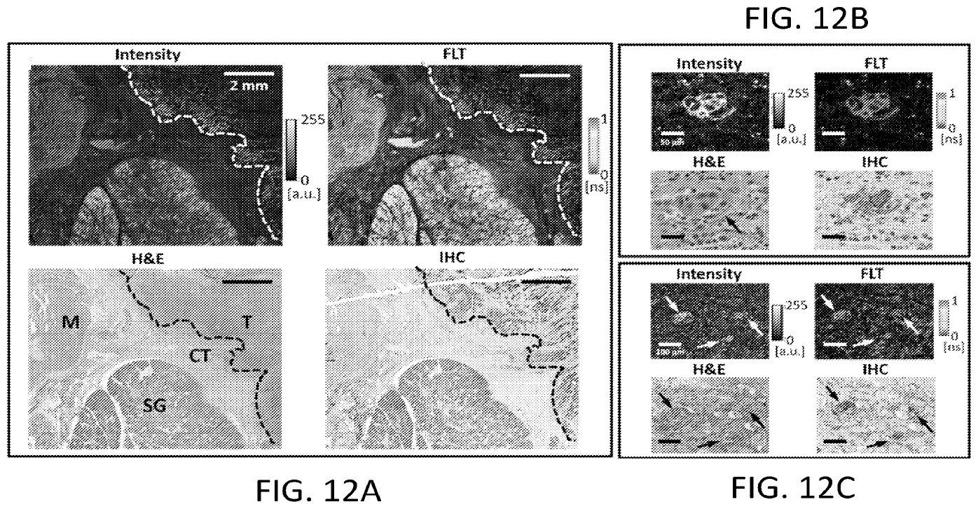

Referring now to FIGS. 12A-12C, some examples of the superior tumor contrast of FLT over fluorescence intensity is shown with respect to tissue regions with strong non-specific uptake of panitumumab-IRDye800CW. In FIG. 12A, the spatial distribution of fluorescence intensity and FLT are shown in a large ROI of the specimen that includes high EGFR expressing tumor regions and low or non-EGFR expressing normal tissue. Corresponding histology and IHC images clearly delineate the tumor boundary (dashed line) from normal salivary glands (SG), muscle (M) and a layer of connective tissue (CT). While fluorescence intensities in tumor, the salivary gland and muscle were comparable, the FLTs in the tumor region are significantly longer than the surrounding normal tissue. IHC images show the highest EGFR expression in tumor followed by the salivary gland and lowest expression in muscle. This trend was closely matched by the FLIM images, which show that the FLTs in the tumor front were significantly longer (0.9-1 ns), compared to the FLTs in the salivary gland (0.7 ns) and muscle (0.5 ns). It is noted that the low uptake and short FLTs in the interior of the tumor in FIG. 12A is due to the presence of non EGFR expressing connective tissue, high density of lymphocytes and a heterogeneous tumor penetration of panitumumab-IRDye800CW. A more homogeneous tumor penetration of panitumumab-IRDye800CW may be achieved using a concurrent loading dose of unlabeled panitumumab with panitumumab-IRDye800CW. The FLTs within the tumor cell clusters are consistently longer than the FLTs of normal tissue, and the areas of long FLT values colocalized with the areas of high EGFR expression.

IN FIG. 12B, a representative ROI is shown with a single cluster of few OSCC cells distant from the primary tumor mass, observed at a higher-magnification (20×). In this ROI, both fluorescence intensity and FLT show high contrast from the tumor cells due to low local non-specific uptake of panitumumab-IRDye800CW. Additionally, FIG. 12C shows an ROI with high non-specific fluorescence with three additional OSCC cell clusters (arrows) surrounded by muscle and lymphocytes. These EGFR overexpressing OSCC cells are distinct only on the FLT image and were hardly distinguishable from the surrounding normal tissue based on fluorescence intensity. All three cell clusters showed average FLTs of 0.96 ns which was significantly longer than the muscle FLT of 0.5 ns. The data indicate that while fluorescence intensity-based imaging identifies certain tumor cell clusters, many tumor cell clusters are indistinguishable from background due to non-specific fluorescence. On the other hand, the FLT of panitumumab-IRDye800CW is consistently longer in tumor cells and is specific to EGFR expression within tumor cells, providing a robust separation of tumor and normal tissue at a microscopic level. It was also confirmed that the longer tumor FLT in the oral cancer specimens does not originate from endogenous tissue autofluorescence.

The FLT images in FIGS. 11A-12C indicate that regions with higher EGFR expression show a longer FLT. While an increased FLTs is expected at individual foci of EGFR binding on the cell membrane or cytoplasm, resolving individual molecules within subcellular compartments is not feasible using confocal imaging. Thus, the FLT at a given pixel of a microscopic image will be the spatial average over intracellular locations that include a range of EGFR expression levels and should correlate with the average EGFR expression within the tissue region corresponding to the pixel.

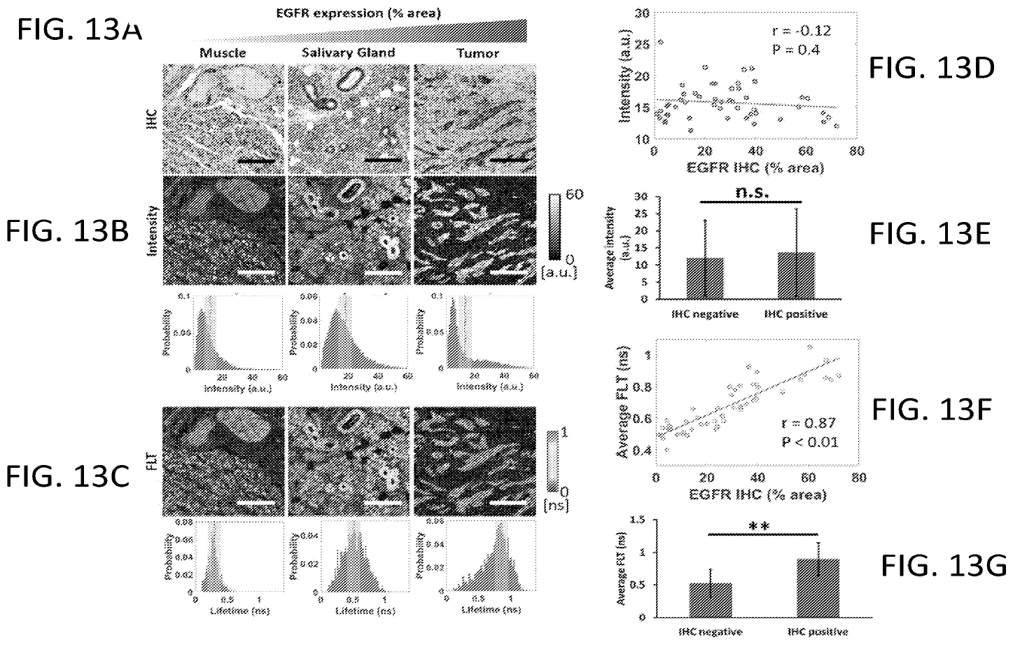

To study this correlation, the relationship between EGFR expression and the mean FLT in multiple tissue sections was quantified. IHC-based quantification of EGFR expression in low, moderate, and high EGFR expressing ROIs revealed that panitumumab-IRDye800CW FLTs correlate strongly with EGFR expression, as shown generally in FIGS. 13A-13G. In particular, FIG. 13A shows IHC of three representative ROIs with increasing EGFR expression, including muscle, salivary glands, and tumor. While panitumumab-IRDye800CW accumulated in EGFR overexpressing tumor cells, there was significant nonspecific uptake in low EGFR expressing muscle and moderate EGFR expressing salivary glands, making the tumor indistinguishable from normal based on fluorescence intensity, as evident from the high overlap of the intensity histograms for the three ROIs, as shown in FIG. 13B. On the other hand, the FLTs of panitumumab-IRDye800CW were shortest in low EGFR expressing muscle region and the longest in high EGFR expressing tumor cells, as shown in FIG. 13C. The average FLTs in the representative muscle, salivary gland and tumor ROIs were 0.4 ns, 0.5 ns and 0.88 ns, respectively. FIG. 13D shows a scatter plot of average fluorescence intensity vs the percent area positive for EGFR in IHC across all the ROIs studied. The scatter plot showed poor correlation (r=−0.12) indicating that panitumumab-IRDye800CW fluorescence intensity cannot reliably be used to quantify EGFR expression in the presence of strong non-specific uptake. Average fluorescence intensities in IHC-confirmed EGFR negative and positive pixels, as shown in FIG. 13E, as obtained from co-registered IHC and FLIM images, were also not statistically different. Average FLTs for each ROI showed a strong positive correlation (r=0.87) with the percent area positive for EGFR in IHC, as shown in FIG. 13F. This indicated that ROIs with higher EGFR expression had longer average FLTs. Additionally, EGFR positive pixels showed significantly longer average FLTs than EGFR negative pixels, as indicated in FIG. 13G. Thus, the results shown in FIGS. 13A-13G confirm that the FLT of panitumumab-IRDye800CW in human tissue is highly correlated with EGFR expression and can provide quantitative estimates of tissue EGFR expression in the presence of strong non-specific uptake.

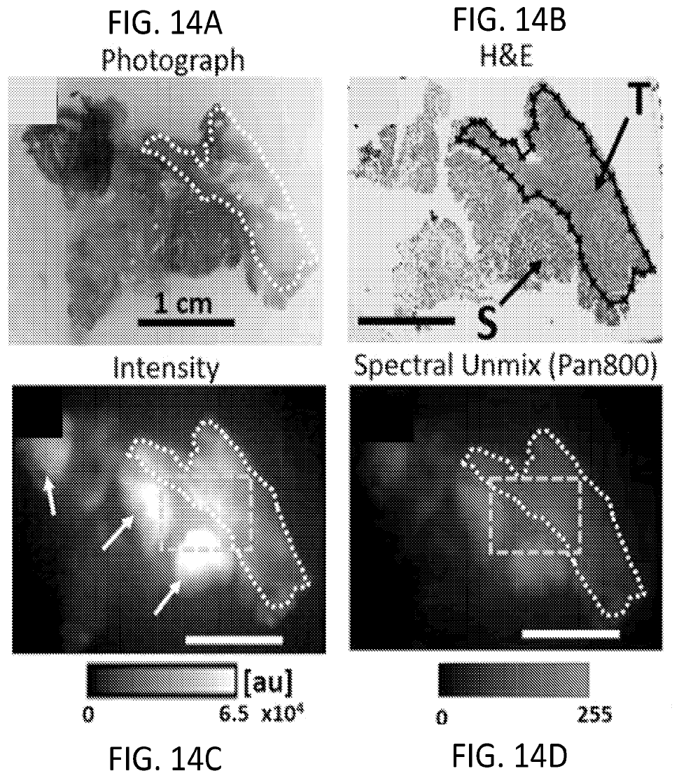

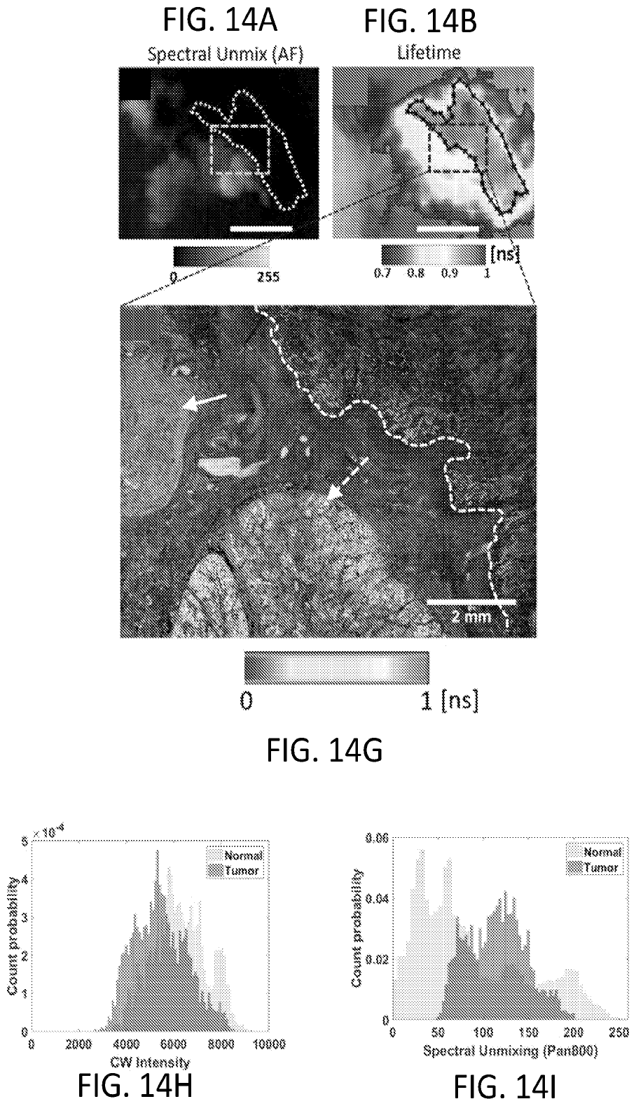

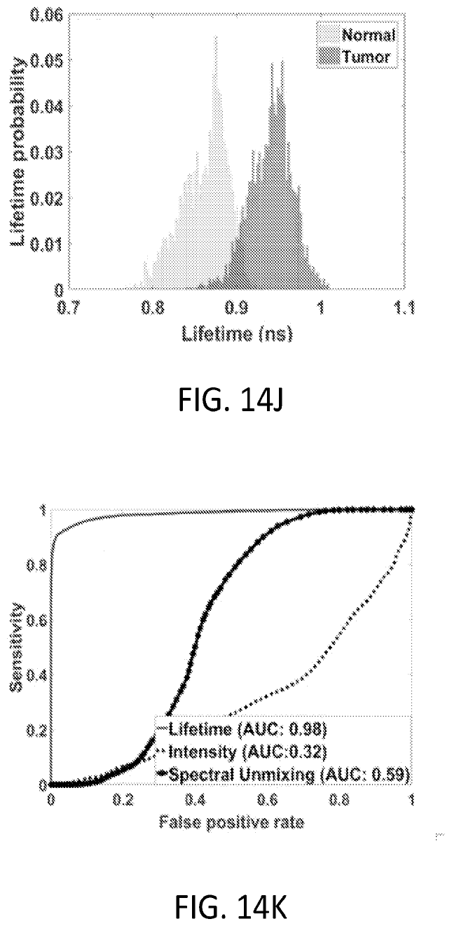

With the cellular specificity of FLT enhancement of panitumumab-IRDye800CW established, we next evaluated the ability of wide-field FLT imaging for tumor-normal classification in thick macroscopic tissue, which is relevant for in situ imaging intraoperatively and for ex vivo imaging of large resection specimens. In particular, FIGS. 14A-14K show widefield time domain (TD) and spectral reflectance imaging of the same tissue block that contained the slides used for the microscopic FLIM and histology analysis shown in FIG. 12A. The color photograph of the specimen in paraffin block (FIG. 14A) was co-registered with histology (FIG. 14B) and the tumor ROI was outlined (black dotted) by pathologists. Widefield fluorescence imaging showed a broad and heterogeneous distribution of fluorescence intensity (FIG. 14C) inside and outside the histologically defined tumor boundary (white dotted), indicating a high level of non-specific fluorescence (white arrows in FIG. 14C) in uninvolved muscle and salivary glands that is nearly indistinguishable from the tumor fluorescence. We verified that spectral unmixing using predetermined tumor and normal spectral basis functions could not clearly distinguish the tumor and normal regions (FIGS. 14D and 14E). However, the FLTs within the tumor region were significantly longer than the FLTs of normal tissue (FIG. 14F), showing little overlap. Histograms of fluorescence intensity, spectral unmixing amplitudes and FLTs within and outside tumor boundary are shown in FIGS. 14H, 14I, and 14J, respectively. The distributions showed highly overlapping fluorescence intensities and spectral amplitudes but distinct FLTs for tumor and normal ROIs with minimal overlap in the corresponding FLT distributions. A receiver operating characteristic (ROC) analysis using the pixel intensity and FLT distributions shown in FIG. 14K, resulted in an area under the curve (AUC) of 0.98 for FLT-based tumor/normal classification compared to an AUC of 0.32 for intensity-based classification (FIG. 14K). While the use of spectral unmixing improved the accuracy over intensity (AUC=0.58), the performance of spectral classification was still significantly poorer than that of FLT imaging. The high accuracy of FLT imaging for tumor normal classification is confirmed by the excellent agreement of the macroscopic FLT imaging-based tumor-normal boundary with the corresponding boundary from the microscopic FLIM image of a thin section of the same slide (FIG. 14G, reproduced from FIG. 12A for ease of comparison) and the histology (FIG. 14B). While the FLT images thus far were obtained using thin tissue slices or in thick excised tissue, detection of FLT changes in vivo in biological tissue in the presence of strong optical absorbing and scattering is also feasible under a wide range of conditions. We have also extensively validated the accuracy of FLT imaging for detecting EGFR over-expressing tumors in vivo using animal tumor models.

The above-described example and results clinically demonstrates that the FLT of a cancer targeted NIR probe systemically injected in patients is longer in cancer cells compared to normal tissue. Further, it shows that the increased tumor FLT of the probe can be exploited to achieve unprecedented accuracy for tumor delineation both at a microscopic level in thin tissue sections and in macroscopic thick tissue specimens, which allows accurate quantification of receptor expression in tissue. This demonstrates cancer specificity at a cellular level in human tissue using exogenous cancer targeted agents.

Besides an improved accuracy for tumor detection, the systems and methods provided herein are also not affected by measurement parameters, such as light illumination power, camera sensitivity, and other system-specific scaling factors. Therefore, FLT can serve as an absolute parameter that can be readily compared across multiple imaging systems and studies, facilitating better standardization in image guided surgery.

The cellular specificity of FLT in cancers has relevance beyond microscopic imaging of thin tissue sections, and can be exploited for imaging tumors in deep tissue. FLT measurements are unaltered by tissue light propagation under a wide range of conditions and can be estimated in the presence of thick tissue without the need for a knowledge of tissue optical properties, which can often be challenging to estimate. Therefore, the cellular specificity of FLT to cancer demonstrates that FLTs measured through thick biological tissue arise solely from tumor cells and not from non-specific probe. This stands in stark contrast to fluorescence intensity, which is strongly attenuated by tissue light propagation (besides its inability to distinguish cancer cell-specific fluorescence from non-specific fluorescence), thereby requiring a full knowledge of tissue optical properties and tissue thickness to accurately quantify probe uptake.

The ability to measure FLTs through deep tissue can be useful when the detection of tumors embedded in thick macroscopic tissue is necessary, such as for the evaluation of margin depth in resection specimens or when imaging deep seated tumors non-invasively in whole organs. FLTs can be detected and localized in deep tissue using tomographic reconstruction algorithms (such as described in U.S. Pat. No. 9,927,362, “System and Methods for tomographic lifetime multiplexing, which is incorporated herein by reference in its entirety). Such methods exploit the relative independence of FLT to tissue scattering and absorption assuming the in vivo FLTs are longer than intrinsic tissue absorption timescales (˜0.2 ns), a condition well satisfied for many NIR fluorophores including IRDye800CW. Thus, the systems and methods provided herein can be extended to create diagnostic systems that quantify cancer-related biomarkers using whole body measurements, or to provide rapid on/off “optical switch” readouts based on predetermined FLT thresholds.

FLT imaging has previously been applied for preclinical studies at the microscopic and whole animal level. While visible FLIM has been evaluated for image guided surgery exploiting endogenous FLTs of tissue components, endogenous FLT contrast between tumor and normal tissue is inherently poor, resulting in low sensitivity and specificity. Further, endogenous fluorescence imaging systems use visible light, which precludes the ability to image sub-surface tumors due to strong tissue attenuation, thereby limiting intraoperative applications to exposed tumors. NIR agents can exploit the greater depth sensitivity of NIR light for intraoperative or deep tissue imaging. In addition, exogenous targeted agents can be used for reporting on molecular expression markers. Nevertheless, endogenous FLIM in the visible spectrum can clearly delineate various tissue structures that could provide valuable morphological information to complement the NIR tumor signal from exogenous agents.

The data described in FIGS. 14A-14K indicate that spectral unmixing techniques can alleviate poor tumor contrast due to non-specific uptake and can be useful when FLT imaging systems are not readily available. The tumor vs normal spectral contrast is still not sufficiently high to provide a tumor detection accuracy comparable to that using FLT. It is noted that spectral contrast is essentially an intensity based measure, and therefore suffers from the same limitations as intensity, such as dependence on measurement parameters, tissue absorption and scattering. Thus, it is harder to quantify in thick biological tissue using such techniques.

The utility and safety of EGFR antibody labelled NIR probes has been extensively studied for OSCC, pancreatic and brain tumors. Although these studies show significant improvement in sensitivity compared to visual identification and palpation, intensity is not always reliable since non-specific uptake in tissue is heterogeneous and can vary across multiple regions within a given specimen. This is clearly illustrated by the above-described data where intensity performs well in some oral tissue regions with good tumor uptake, while high non-specific uptake is present in other areas of oral tissue such as salivary glands, significantly diminishing tumor contrast. FLTs of tumors, on the other hand are consistently and uniformly longer than normal oral tissue and therefore provide a robust measure of tumor uptake.

Since panitumumab-IRDye800CW has been extensively tested for safety in humans and intraoperative FLT imaging has been demonstrated to be clinically feasible, the results presented here have immediate clinical relevance for intraoperative surgical guidance in EGFR over-expressing cancers. Over 90% of head and neck cancers over-express EGFR. Besides the multiple clinical trials of anti-EGFR antibody labelled probes for OSCC, clinical trials of EGFR-antibody-based probes have been conducted in brain, colorectal, and pancreatic cancers. FLT imaging using panitumumab-IRDye800CW is therefore likely to strongly impact surgical guidance for these cancers as well. In addition to EGFR targeting, FLT contrast can also benefit tumor imaging using other receptor targeted probes. Early preclinical studies have shown tumor specific FLT changes of fluorescently labelled affibody for human epidermal growth factor receptor-2 (HER-2) in mice. Also, probes targeted to immune expression markers exhibit longer lifetimes in tumor cells compared to normal tissue. As new probes to target cancer-specific molecular markers continue to be developed, the systems and methods provided herein can be applied to these newly developed agents as well. Given its powerful and unique benefits, FLT imaging using targeted molecular imaging agents can an important role in a wide range of clinical settings ranging from cancer diagnostics to surgical therapy.

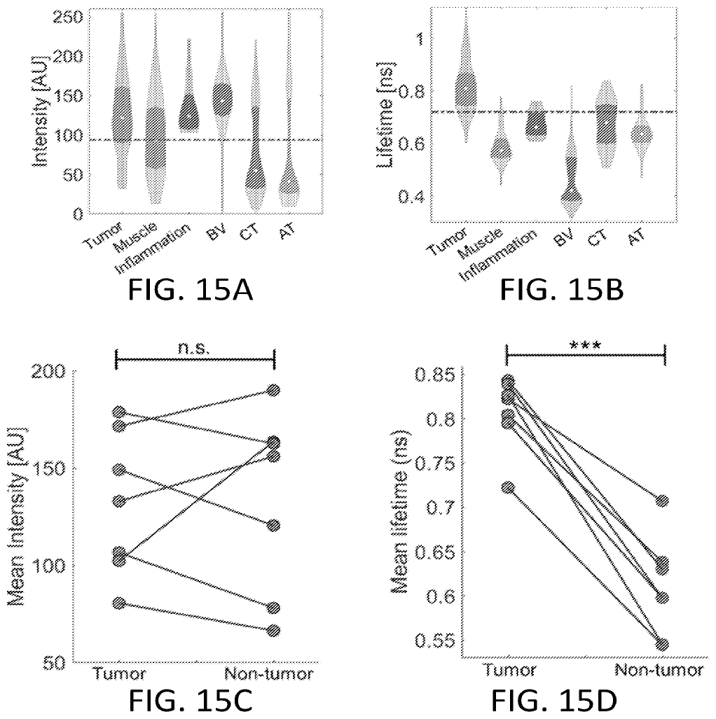

FIGS. 15A-15D are a series of graphs showing fluorescence lifetime enhancement in sarcoma tumors. The violin plots of FIGS. 15A and 15B show the distribution of fluorescence intensity, (FIG. 15A), and FLT, (FIG. 15B), in sarcoma tumors and several normal tissue types resected from multiple patients (n=10) injected with ICG between 0 h and 48 h prior to surgery. Here, BV refers to blood vessels; CT refers to connective tissue; and AT refers to adipose tissue. Mean fluorescence intensity of tumor and normal tissue across seven patients is shown in the graph of FIG. 15C and the corresponding FLTs of tumor and normal tissue for seven patients is shown in FIG. 15D, wherein the circles on the left illustrate tumor values and normal tissue is represented in the circles on the right. Means were calculated for multiple ROIs (>20) of histologically identified tumor or normal tissue for each patient. Dashed lines in FIG. 15A and FIG. 15B represent the threshold intensity or FLT that provide the highest sensitivity and specificity. Mann-Whitney U test (two-tailed): *** p<0.001.

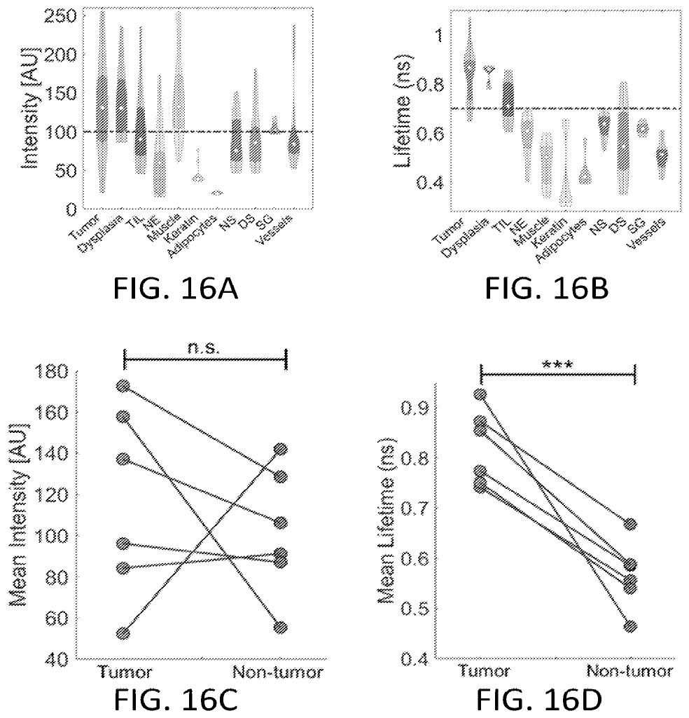

FIGS. 16A-16D are a series of graphs showing fluorescence lifetime enhancement in head and neck cancers. FIGS. 16A and 16B are violin plots showing the distribution of fluorescence intensity (FIG. 16A), and FLT (FIG. 16B), in head and neck cancers and various normal oral tissue types across multiple patients (n=8) injected with indocyanine Green (ICG) at least 24 hours prior to surgery. Here, NE refers to normal epithelium; NS refers to normal stroma; DS refers to desmoplastic stroma; and SG refers to salivary glands. The mean fluorescence intensity across 6 patients is shown in FIG. 16C, and the FLT is shown in FIG. 16D, where the tumor is reflected in the circles on the left of each graph and normal tissue is reflected on the circles on the right. The filled circles, themselves, represent the mean of multiple ROIs (>20) of histologically identified tumor or normal tissue for each patient. Mann-Whitney U test (two-tailed): *** p<0.001.

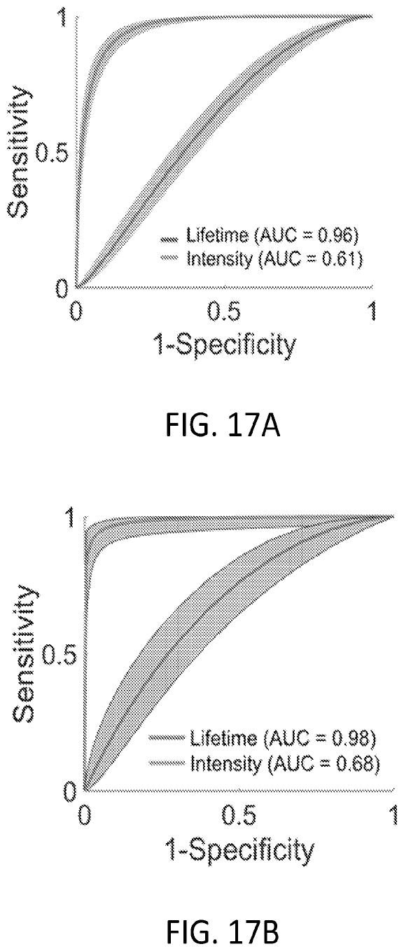

FIG. 17A is a graphs of receiver operating characteristic (ROC) plot of sensitivity vs. false positive rate (1—specificity) for fluorescence lifetime (FLT) and intensity based tumor versus normal classification in specimens across 10 sarcoma patients. FIG. 17A shows an accuracy (measured as area under the curve (AUC)) of 0.96 and 0.56, respectively. FIG. 17B is a similar ROC plot across 8 head and neck cancer patients showed an accuracy (AUC) of 0.96 and 0.61 for FLT and intensity based tumor vs normal classification, respectively.

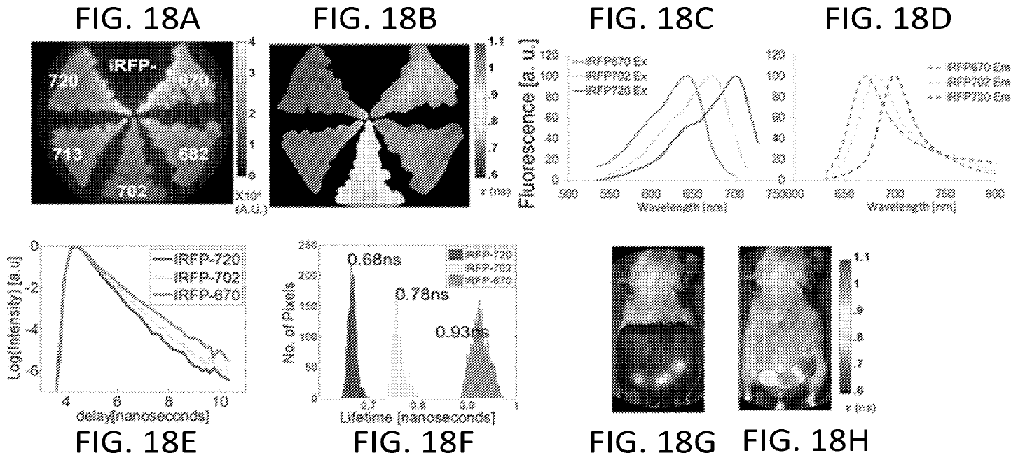

FIGS. 18A-18H are graphs and images illustrating in vitro and in vivo lifetime multiplexing of near infrared fluorescent proteins (iRFPs). All fluorescence data was acquired with single excitation/emission filter pair: ex: 650/40 nm, em: 700 nm long pass. FIG. 18A is an image of fluorescence intensity of bacteria expressing five iRFP variants (iRFP670, 682, 702, 713 and 720) and FIG. 18B is a fluorescence lifetime image of the same. FIG. 18C shows the normalized excitation and FIG. 18D shows the emission spectra of iRFP670, 702 and 720. FIG. 18E is a graph of time domain (TD) fluorescence signal of iRFP670, 702 and 720 in bacteria. FIG. 18F is a histogram of lifetimes derived from FIG. 18B. FIG. 18G is an image showing fluorescence intensity and FIG. 18H is an image showing fluorescence lifetime of three MTLn3 tumors expressing iRFP670, 702 and 720 located in the mammary fat pad of a female nude mouse.

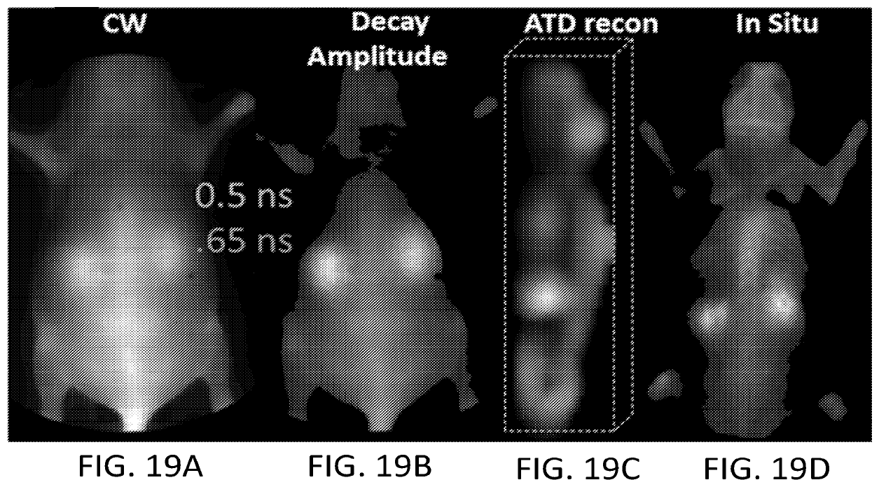

FIGS. 19A-19D provide images of a mice using intensity imaging (FIG. 19A) and tomographic fluorescence lifetime multiplexing of anatomically targeted fluorophores (FIGS. 19B-D). The mice in the images of FIGS. 19A-19D were injected i.v. with Osteosense800 (0.65 ns, targeting the skeletal system) and ZE169 (0.5 ns, targeting kidneys.) The cancer is clear in FIGS. 19B-19D, but difficult to discern in FIG. 19A.

The present invention may be embodied in other specific forms without departing from its spirit or essential characteristics. The described implementations are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method of assessing tissue to determine a presence or absence of cancer cells, the method comprising:
acquiring fluorescence lifetime (FLT) data from tissue having received a fluorescent compound;
processing the FLT data to determine a FLT signal at each of a plurality of locations across the tissue;
determining FLT data at any of the plurality of locations above a threshold indicative a presence of cancer cells; and
generating a report indicating any of the plurality of locations above the threshold as indicative the presence of cancer cells.

2. The method of claim 1, wherein acquiring the FLT data includes acquiring fluorescence data from the tissue at a sequence of consecutive time points starting from a chosen time origin to a chosen end point.

3. The method of claim 2, wherein the FLT data is given by:
$${y}^{TD} ( t ) = {a}_{0} {e}^{- t / τ}$$
where y^{TD} (t) is time domain data as a function of time, t, between the chosen time origin and the chosen end point, α₀ is a decay amplitude, which is related to fluorophore concentration, quantum yield, and experimental scaling constants, and t is a lifetime of a fluorophore applied to the tissue to elicit the FLT data.

4. The method of claim 3, wherein the FLT data is linearized based on logarithmic or series expansion, and the resulting linearized data is fit for the lifetimes using a linear least squares based approach.

5. The method of claim 1, wherein acquiring the FLT data includes acquiring fluorescence data cumulatively from the tissue beginning at a chosen time origin to an end point or to multiple time points in time.

6. The method of claim 5, wherein the FLT data is given by:
$${y}^{QTD} ( t ) = {\int }_{0}^{t} y ( {t}^{′} ) {dt}^{′}$$
where y^{QTD} is the quasi time domain data as a function of time, t, from the chosen time origin to the end point or multiple points in time.

7. The method of claim 5, wherein the FLT data is given by:
$${y}^{QTD} ( t ) = {\int }_{0}^{t} y ( {t}^{′} ) {dt}^{′} {y}^{QTD} ( t ) = {a}_{0} {\int }_{0}^{t} {e}^{- \frac{{t}^{′}}{τ}} {dt}^{′} = {a}_{0} τ ( 1 - {e}^{- \frac{t}{τ}} )$$
y^{QTD} is the quasi time domain data as a function of time, t, from the chosen time origin to the chosen end point or multiple points in time, with the FLT data fit to a function, α₀τ(1−e^{-t/τ}), where t is the lifetime of a fluorophore applied to the tissue to elicit the FLT data and α₀ is a decay amplitude, which is related to fluorophore concentration, quantum yield, and experimental scaling constants.

8. The method of claim 7, wherein the FLT data is linearized based on logarithmic or series expansion, and the resulting linearized data is fit for the lifetimes using a linear least squares based approach.

9. The method of claim 7, further comprising using continuous wave (CW) fluorescence intensity data and a single time gate to generate an FLT image using:
$$τ = - \frac{{T}_{1}}{log [ 1 - \frac{{y}^{QTD} ( {T}_{1} )}{{y}^{QTD} ( ∞ )} ]} ;$$
where y^{QTD}(T₁)=∫₀^{T}^{₁}α₀e^{-t/τ}dt=α₀τ(1−e^{-τ}^{₁}^{/τ}) is the FLT data acquired with a gate width T₁.

10. The method of claim 1, wherein acquiring the FLT data includes acquiring fluorescence data from tissue at a single or multiple modulation frequency and phases.

11. The method of claim 1, wherein the threshold is a cutoff lifetime selected for one of a given cancer type or an anatomical region.

12. The method of claim 1, wherein generating the report includes producing an overlay of FLT data spatially registered to the tissue.

13. The method of claim 12, wherein the overlay includes a mask defining a percentage certainty of cancer or a color coding showing the FLT data as a lifetime map.

14. The method of claim 1, wherein generating the report includes producing a lifetime histogram.

15. The method of claim 1, wherein acquiring the FLT data includes arranging an optical probe and the tissue in proximity to acquire the FLT data.

16. The method of claim 1, wherein the tissue is located in an in vivo surgical site.

17. A medical imaging system comprising:
an optical source configured to deliver light to tissue;
a detector configured to receive light fluoresced by the tissue and produce fluorescence lifetime (FLT) data;
a processor configured to:
analyze the FLT data to determine a presence or absence of cancer in the tissue;
generate a report indicating a spatial location of any cancer determined as present in the tissue; and
a display configured to display the report to guide a surgical procedure to remove the cancer.

18. The system of claim 17, wherein the processor is further configured to analyze the FLT data to determine a FLT signal at each of a plurality of locations across the tissue and determine FLT data at any of the plurality of locations above a threshold indicative a presence of cancer cells to analyze the FLT data.

19. The system of claim 18, where wherein the threshold is a cutoff lifetime selected for one of a given cancer type or an anatomical region.

20. The system of claim 17, wherein the processor is further configured to generate the report by producing an overlay of FLT data spatially registered to the tissue.

21. The system of claim 20, wherein the overlay includes a mask defining a percentage certainty of cancer or a color coding showing the FLT data as a lifetime map.

22. The system of claim 17, wherein the processor is further configured to generate the report by producing a lifetime histogram.

23. The system of claim 17, wherein the processor is configured to assemble the FLT data by receiving fluorescence data cumulatively from the detector beginning at a chosen time origin and continuing to an end point or to multiple time points in time.

24. The system of claim 17, wherein the FLT data is given by:
$${y}^{TD} ( t ) = {a}_{0} {e}^{- t / τ}$$
where y^{TD} (t) is time domain data as a function of time, t, which can assume values that range from a chosen time origin to an end point, and α₀ is a decay amplitude, which is related to fluorophore concentration, quantum yield, and experimental scaling constants.

25. The system of claim 17, wherein the FLT data is given by:
$${y}^{QTD} ( t ) = {\int }_{0}^{t} y ( {t}^{′} ) {dt}^{′}$$
where y^{QTD} is the quasi time domain data as a function of time, t, from a chosen time origin to an end point or multiple points in time.

26. The system of claim 17, wherein the FLT data is given by:
$${y}^{QTD} ( t ) = {a}_{0} {\int }_{0}^{t} {e}^{- \frac{{t}^{′}}{τ}} {dt}^{′} = {a}_{0} τ ( 1 - {e}^{- \frac{t}{τ}} )$$
y^{QTD} is the quasi time domain data as a function of time, t, from a chosen time origin to an end point or multiple points in time, with the FLT data fit to a function, τ(1−e^{-t/τ}), where τ is a lifetime of a fluorophore applied to the tissue to elicit the FLT data.

27. The system of claim 17, further comprising a sample chamber configured to receive an ex vivo sample for data acquisition.

28. The system of claim 17, wherein the detector is further configured to receive light fluoresced by the tissue and produce intensity data.
